(19) United States
(12) Patent Application Publication
(10) Pub. No.: US 2025/0032311 A1
(43) Pub. Date: Jan. 30, 2025

(54) DEVICE FOR REVERSIBLY BLOCKING ACTIVITIES OF TARGET REGION AND APPLICATIONS OF SAME

(71) Applicants: NORTHWESTERN UNIVERSITY, Evanston, IL (US); WASHINGTON UNIVERSITY, St. Louis, MO (US)

(72) Inventors: John A. Rogers, Wilmette, IL (US); Jonathan T. Reeder, Eugene, OR (US); Matthew MacEwan, Webster Groves, MO (US); Wilson Z. Ray, St. Louis, MO (US)

(21) Appl. No.: 18/716,989

(22) PCT Filed: Dec. 9, 2022
(86) PCT No.: PCT/US2022/052342

§ 371 Date: Jun. 6, 2024

Related U.S. Application Data
(60) Provisional application No. 63/288,058, filed on Dec. 10, 2021.

(51) Int. Cl. A61F 7/12; A61F 7/00

(52) U.S. Cl. CPC A61F 7/12; A61F 2007/0058; A61F 2007/126

(57) ABSTRACT

A device for reversibly blocking activities of a target region of a subject includes a microfluidic system configured to route a fluid around the target region to change a local temperature of the target region; and an electronic system coupled with the microfluidic system for providing a real-time feedback.

### CROSS-REFERENCE TO RELATED PATENT APPLICATION

This application claims priority to and the benefit of U.S. Provisional Application Ser. No. 63/288,058, filed Dec. 10, 2021, which is incorporated herein in its entirety by reference.

### STATEMENT AS TO RIGHTS UNDER FEDERALLY-SPONSORED RESEARCH

This invention was made with government support under Grant No. CMMI1635443 awarded by the National Science Foundation. The government has certain rights in the invention.

### FIELD OF THE INVENTION

The present invention relates generally to biosensors, and more particularly to soft, bioresorbable, evaporative microfluidic coolers for reversible conduction block of peripheral nerves and applications of same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The high therapeutic efficacy of opioids has led to their widespread use despite increasing rates of addiction and opioid-related deaths due to overdose. In 2017, opioid use disorder affected 2.1 million Americans, caused 47,885 fatal overdoses, and led to economic costs in excess of $1 trillion USD. Provisional data from the Centers for Disease Control indicate that annual opioid overdose deaths increased 37% during 2020 from 50,178 to 68,821. This significant and increasing societal burden motivates the development of localized, non-opioid, and non-addictive pain management techniques. Miniaturized implantable devices that eliminate pain signals locally in peripheral nerves suggest a potential role for engineering-based treatments that avoid side effects associated with opioids and other analgesics. The controlled input of electrical, pharmacological, optical, mechanical, or thermal stimuli to neural tissue can lead to local and reversible neural blocking. For example, mechanical forces induced by targeted ultrasonic energy can regulate peripheral nerve activity, but stimulation parameters that provide therapeutic benefit, yield acceptable safety profiles, and offer compatibility with methods of monitoring the block have yet to be demonstrated. Optical activation of genetically modified cell populations enables targeted and temporally precise nerve inactivation, but requires safe and effective gene therapy vectors. Local delivery of pharmacological agents such as opioids directly to the spinal cord or nerves via implanted drug pumps represents an alternative but retains risks of dependence and withdrawal. Recent reports describe implantable drug delivery devices for the central and peripheral nervous systems. Here, the response time of blocking is on the order of 30 min to two hours as largely dictated by diffusion-dominated pharmacokinetics. Most applications focus on treatments of chronic pain or other applications that demand persistent block. Electrical stimulation represents the most successful scheme for local pain management, as evidenced by clinical deployment in the deep brain, spinal cord, and peripheral nerves. This approach, however, has side effects such as onset response, manifested by pain during initiation of the block, and sustained paresthesia. Also, electrical artifacts associated with stimulation can complicate simultaneous recording of spontaneous and evoked neural activity and thus real-time evaluation of neural function.

Temperature modulates neural activity, as with all biological functions, through purely physicochemical relationships. Metabolic, electrogenic, and ionic activity in neural tissue all exhibit, to some approximation, temperature dependence that follows the Arrhenius equation. Temperature changes on the order of 10° C. or greater can strongly modulate neural activity in the central and the peripheral nervous systems. Local cooling of peripheral nerves decreases conduction velocity and signal amplitude of neural activity. Blocking of transmission of compound action potentials in mammalian nerves occurs below 15° C. Cooling applied to peripheral nerves is a promising approach for blocking pain signals because it is non-addictive, rapidly reversible, can be applied locally, avoids any onset response, and allows for simultaneous electrical interrogation of the blocked nerve.

Analgesic nerve cooling requires spatiotemporally-precise control of temperature to maximize desired outcomes and to mitigate side effects. The delivery of pre-cooled fluid through looped metal or silicone tube interfaces to ex vivo nerve preparations, and in a few cases in awake, freely moving animals shows some promise. Here, reductions in temperature localized at the anatomical site of interest follow from actively controlled mass transfer of liquid coolant to this region through systems with thermal insulation to mitigate parasitic thermal losses along the fluidic delivery pathway. As an alternative, thermoelectric modules can apply cooling power directly at a desired location, as demonstrated with tissues of the cortex and sub-cortical structures. Rigid and bulky form factors, high power requirements, and the production of waste heat preclude use in peripheral nerves of freely moving animals. As a result, these and other mechanisms cannot provide precise cooling to small volumes of neural tissue in formats compatible with long term implantation. In addition, all previously reported technologies require surgical extraction after a period of need. These engineering challenges currently prevent the use of local cooling as a practical approach for peripheral nerve pain management.

Therefore, a heretofore unaddressed need exists in the art to address the aforementioned deficiencies and inadequacies.

### SUMMARY OF THE INVENTION

In light of the foregoing, this invention discloses soft, bioresorbable, microfluidic devices that enable delivery of focused, minimally invasive cooling power at arbitrary depths in living tissues with real-time temperature feedback control.

In one aspect, the invention relates to a device for reversibly blocking activities of a target region of a subject comprising a microfluidic system configured to route a fluid around the target region to change a local temperature of the target region so as to reversibly block activities of the target region.

In one embodiment, the microfluidic system is operably in communication with the target region.

In one embodiment, the microfluidic system utilizes a liquid to gas phase transition as a cooling mechanism to change the local temperature of the target region.

In one embodiment, the microfluidic system comprises at least one fluidic chamber formed in a microfluidic layer.

In one embodiment, the at least one fluidic chamber has a length, a footprint and a volume, wherein the length defines a coverage angle of the microfluidic system when wrapping around the target region.

In one embodiment, a cooled area in the target region is confined predominately to a surface directly associated with the at least one fluidic chamber.

In one embodiment, the microfluidic system further comprises transcutaneous colinear interconnects that deliver the fluid to the at least one fluidic chamber in a completely sealed system that provides fluidic access at the ends.

In one embodiment, the microfluidic system further comprises at least first and second input channels and at least one output channel fluidically connected to the at least one fluidic chamber.

In one embodiment, the at least one output channel is colinear to the at least first and second input channels.

In one embodiment, the first and second input channels have widths in ranges of about 50-150 μm and about 200-600 μm, respectively, and the output channel has a width in a range of about 200-600 μm.

In one embodiment, the fluid comprises a coolant and a dry gas being operably transported into the at least one fluidic chamber via the first and second input channels, respectively.

In one embodiment, the microfluidic system is configured such that a simultaneous initiation of the coolant and the dry gas flows into the at least one fluidic chamber prompts evaporation of the coolant at a microfluidic junction between the first and second input channels of the coolant and the dry gas and along the at least one fluidic chamber.

In one embodiment, the at least one fluidic chamber comprises at least one serpentine microfluidic channel formed with a plurality of U-shaped turns over a region in the microfluidic layer.

In one embodiment, the at least one serpentine microfluidic channel operably routes a volume of the coolant to the target region where a flow of the dry gas triggers local and fully contained evaporation of the coolant.

In one embodiment, mass flow rates of the coolant and the dry gas, and the length, the footprint and the volume of the at least one fluidic chamber determine magnitude and localization of the cooling effect.

In one embodiment, the coolant is fluorocarbons, and wherein the dry gas comprises any dry gas including N₂, CO₂, argon, or mixtures thereof.

In one embodiment, the coolant is a bioinert coolant including perfluoropentane (PFP).

In one embodiment, the coolant is a non-bioinert coolant including diethyl ether.

In one embodiment, the microfluidic system further comprises at least one first pump in fluidic communication with the at least first and second input channels for delivering the fluid to the at least one fluidic chamber to change the local temperature of the target region.

T In one embodiment, the microfluidic system further comprises at least one second pump in fluidic communication with the at least one output channel for withdrawing the fluid from the at least one fluidic chamber.

In one embodiment, the device is configured such that a phase change prompts a temperature of the device in a planar, uncurled configuration to drop to about −20° C. within about 2 min or less after initializing flow in ambient, room temperature conditions.

In one embodiment, the microfluidic system is formed of a bioresorbable elastomer.

In one embodiment, the bioresorbable elastomer is selected to have an elastic modulus that is compatible with the target region, with a controllable rate of degradation via surface erosion.

In one embodiment, the elastic modulus is in a range of about 10 kPa and 1 GPa.

In one embodiment, the bioresorbable elastomer comprises biopolymers, synthetic polymers, proteins, polysaccharides, poly(octanediol citrate) (POC), polydioxanone, poly(lactic-co-glycolic acid) (PLGA), polylactic acid (PLA), collagen, chitosan, poly(glycerol sebacate), poly(lactic-co-glycolic acid), poly(caprolactone), poly(octamethylene maleate (anhydride) citrate, poly(itaconate-co-citrate-co-octanediol).

In one embodiment, the microfluidic system is formed of a non-bioresorbable elastomer.

In one embodiment, the non-bioresorbable elastomer comprises thermoplastic elastomers, styrenic materials, olefenic materials, polyolefin, polyurethane thermoplastic elastomers, polyamides, synthetic rubbers, polydimethylsiloxane, polybutadiene, polyisobutylene, poly(styrene-butadiene-styrene), polyurethanes, polychloroprene, silicones, or a combination thereof.

In one embodiment, the device further comprises an electronic system coupled with the microfluidic system for providing a real-time feedback.

In one embodiment, the electronic system comprises at least one sensor configured to detect a temperature of the target region for providing a temperature feedback.

In one embodiment, the at least one sensor is a flexible temperature sensor.

In one embodiment, the at least one sensor is a bioresorbable, resistance-based temperature sensor.

In one embodiment, the at least one sensor comprise a magnesium resistance-based temperature sensor.

In one embodiment, the magnesium resistance-based temperature sensor comprises a serpentine magnesium trace.

In one embodiment, the at least one sensor comprises a layer of magnesium (Mg) encapsulated with silicon dioxide (SiO₂) and supported by a cellulose acetate substrate.

In one embodiment, the at least one sensor comprises a multilayered stack of SiO₂/Mg/SiO₂ deposited on a cellulose acetate substrate.

In one embodiment, the at least one sensor is encapsulated by two layers of the bioresorbable elastomer of POC.

In one embodiment, the electronic system operably lies coplanar with the microfluidic system, with the at least one temperature sensor at the distal end of the device.

In one embodiment, the target region comprises soft tissue structures including a peripheral nerve.

In one embodiment, the electronic and microfluidic systems terminate in a cuff structure with a diameter matched to the peripheral nerve, to provide an intimate mechanical and thermal interface to the nerve, without the need for sutures.

In one embodiment, the device further comprises a collection member coupled to the microfluidic system for condensing and collecting gaseous coolant.

In one embodiment, the collection member comprises a condenser connected to the at least one output channel of the microfluidic system, and a liquid trap connected to the condenser.

In one embodiment, the collection member further comprises a pressure meter connected at a point between the output channel of the microfluidic system and an inlet to the condenser for measuring backpressure of the condenser.

In one embodiment, the device is configured to allow for long-lived, biocompatible, and intimate mechanical and thermal interfaces to peripheral nerves.

In one embodiment, the device is capable of spatiotemporally precise, large-amplitude cooling with integrated sensing capabilities for closed-loop control.

In one embodiment, the device is configured to deliver focused, minimally invasive cooling power at arbitrary depths in living tissues with real-time temperature feedback control.

In one embodiment, the device is usable for a treatment of pain, without a need for pharmaceutical interventions.

In one embodiment, the device is implantable device.

In one embodiment, the device is a hybrid wearable/implantable device

In one embodiment, the device is biocompatible.

In one embodiment, the device is constructed entirely with water-soluble constituent materials that are controllably dissolvable in biofluids and are subsequently bioresorbable therein.

In another aspect, the invention relates to a method for reversibly blocking activities of a target region of a subject, comprising routing a fluid around the target region to change a local temperature of the target region so as to reversibly block activities of the target region.

In one embodiment, said the fluid around the target region is performed by a microfluidic system that utilizes a liquid to gas phase transition as a cooling mechanism to change the local temperature of the target region.

In one embodiment, the microfluidic system comprises at least one fluidic chamber formed in a microfluidic layer.

In one embodiment, the microfluidic system further comprises at least first and second input channels and at least one output channel fluidically connected to the at least one fluidic chamber.

In one embodiment, the fluid comprises a coolant and a dry gas being operably transported into the at least one fluidic chamber via the first and second input channels, respectively.

In one embodiment, the microfluidic system is configured such that a simultaneous initiation of the coolant and the dry gas flows into the at least one fluidic chamber prompts evaporation of the coolant at a microfluidic junction between the first and second input channels of the coolant and the dry gas and along the at least one fluidic chamber.

In one embodiment, the at least one fluidic chamber comprises at least one serpentine microfluidic channel formed with a plurality of U-shaped turns over a region in the microfluidic layer.

In one embodiment, the at least one serpentine microfluidic channel operably routes a volume of the coolant to the target region where a flow of the dry gas triggers local and fully contained evaporation of the coolant.

In one embodiment, the method further comprises measuring a temperature of the target region to provide a real-time feedback.

In one embodiment, said measuring the temperature of the target region is performed by a flexible temperature sensor.

In one embodiment, the flexible temperature sensor is bonded to the microfluidic system.

In yet another aspect, the invention relates to a method for fabricating a device for reversibly blocking activities of a target region of a subject, comprising fabricating a microfluidic system for routing a fluid around the target region to change a local temperature of the target region; and fabricating an electronic system coupled with the microfluidic system for providing a real-time feedback.

In one embodiment, said fabricating the microfluidic system comprises providing a patterned substrate; coating a sacrificial layer on the patterned substrate; casting a prepolymer on the coated sacrificial layer and subsequently curing the casted prepolymer to form a polymer layer; and removing the sacrificial layer from the polymer layer to define at least one fluidic chamber with at least one at least one serpentine microfluidic channel.

In one embodiment, the sacrificial layer is formed of poly(acrylic acid) (PAA).

In one embodiment, the prepolymer is a poly(octanediol citrate) (POC) prepolymer.

In one embodiment, said fabricating the electronic system comprises sequentially depositing SiO₂, Mg and SiO₂ onto cellulose acetate to form a temperature sensor layer; and encapsulating the temperature sensor layer with top and bottom layers of POC.

In one embodiment, the method further comprises bonding the electronic system to the microfluidic system.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIGS. 1A-1F show soft, bioresorbable, evaporative microfluidic coolers for an on-demand nerve block, according to embodiment of the invention. FIG. 1A: Transmission of postoperative acute pain signals through peripheral nerves. FIG. 1B: Local cooling of peripheral nerves provides an on-demand nerve block. FIG. 1C: Treatment termination and cooler bioresorption after completion of the healing process. FIG. 1D: A soft, bioresorbable nerve-cooling system that features elastomeric interconnects and a terminal cuff structure. FIG. 1E: A soft, curled, nerve-clasping structure provides secure attachment to nerves without sutures. FIG. 1F: Construction from water-soluble materials enables device dissolution and subsequent bioresorption (PBS at 75° C., pH 7.4).

FIGS. 2A-2F show an evaporative microfluidic cooling and temperature sensing system, according to embodiment of the invention. FIG. 2A: Exploded device render showing layers of the microfluidic and electronic systems. FIG. 2B: Schematic illustration of the microfluidic and electronic systems design. FIG. 2C: A flow of dry N₂ prompts the complete evaporation of PFP within three serpentine turns at low molar ratio (X_{PFP}=0.1) inside the serpentine microfluidic channel. FIG. 2D: Increasing the PFP molar fraction (X_{PFP}=0.5) extends the evaporation throughout the length of the serpentine microfluidic channel. FIG. 2E: Evaporative microfluidic cooling prompts reduction of the temperature of the device surface to −20° C. in ambient conditions, as shown by thermal imaging. FIG. 2F: A bioresorbable electronic system for providing nerve-temperature feedback consists of a magnesium resistance-based temperature sensor on a cellulose acetate (CA) substrate.

FIGS. 3A-3I show in vitro studies of nerve-cooling efficacy according to embodiment of the invention. FIG. 3A: The N₂ flow rate depends on N₂ pressure (P_{N2}) and PFP flow rate. Q_{N2}, N₂ flow rate; QPFP, PFP flow rate. FIG. 3B: Phantom-nerve temperature depends on PFP and N₂ flow rates. FIG. 3C: A PFP molar fraction of 0.13 produces the lowest nerve temperature (−1.4° C.). Qₜₒₜₐₗ. Total flow rate. FIG. 3D: Phantom-nerve cooling rates greater than 3° C./s are prompted by initiation of a 300 ml/min flow of PFP. FIG. 3E: Systematic reduction in PFP flow rate from 300 ml/min enables controlled rewarming of the nerve over a 20 min period. FIG. 3F: Evaporative microfluidic cooling enables precise and stable nerve cooling for more than 15 min. FIG. 3G: The effect of a convective environment on the ability to cool a phantom nerve, as shown by a comparison of 37° C. water and hydrogel baths. FIG. 3H: Physiologically relevant rat sciatic nerve blood flow rates induce an increase in nerve temperature during cooling from 3° to 5° C. in a phantom nerve. Error bars represent standard deviation of three trials. FIG. 3I: POC microfluidics provide precise nerve cooling for 21 days in vitro (PBS, 37° C., pH 7.4).

FIGS. 4A-4I show cooling localization according to embodiment of the invention. FIGS. 4A-4C: Illustrations of the experimental setup for quantifying cooling localization (FIG. 4A) radially, (FIG. 4B) longitudinally, and (FIG. 4C) across the surface of the nerve. FIGS. 4D-4F: A thermochromic (TC) hydrogel visually indicates the 10° C. thermocline for a plane (FIG. 4D) perpendicular to the nerve, (FIG. 4E) parallel to the nerve, and (FIG. 4F) above a flat, uncurled cooler. FIGS. 4G-4I: Simulations of the cases shown in FIGS. 4A-4C, respectively, starting at the bottom of the TC hydrogel (z=0).

FIGS. 5A-5F show cooling-induced nerve block and analgesia according to embodiment of the invention. FIG. 5A: Amplitude of muscular contraction in the tibialis anterior, as measured by EMG, diminishes with reduced temperature, and signal latency increases and subsequently recovers after rewarming. FIG. 5B: Amplitude of CNAP diminishes with reduced temperature, and signal latency increases and subsequently recovers upon rewarming. FIG. 5C: Illustration of the bioresorbable nerve cooler interfacing with the sciatic nerve of a rat. FIG. 5D: Illustration of the subcutaneous routing path of the bioresorbable device and nonbioresorbable interconnects. A SNI generates persistent touch sensitivity of the paw and serves as a model for neuropathic pain. FIG. 5E: Image of a freely moving rat in a test environment for quantifying the mechanical nociceptive threshold. FIG. 5F: Cooling the sciatic nerve to 10° C. increases the mechanical nociceptive threshold by a factor of seven in animals that received both a SNI and cuff, indicating a significant cooling-induced analgesic effect. Changes in mechanical sensitivity of the contralateral for the same animals are insignificant. Error bars represent standard error of the mean. ns, not significant; * P<0.05.

FIG. 6 shows schematically a fabrication process for bioresorbable hybrid microfluidic-electronic nerve coolers according to embodiment of the invention.

FIG. 7 shows a bioresorbable temperature sensor according to embodiment of the invention. Panel A: Micrographs of the bioresorbable temperature sensor. Panel B: Temperature calibration curves for three devices. The room temperature resistance of three devices is 915±90Ω. Each exhibits a linear change in resistance with temperature, with a sensitivity of 2.05±0.13 Ω/° C. from 0 to 40° C. Panel C: Encapsulating layers of POC and SiO₂ protect the Mg and prevent its hydrolysis for greater than 2 h during complete immersion in PBS (pH 7.4) at 37° C. As demonstrated in FIGS. 3A-3I, the precise and stable evaporative cooling provided by the device obviates the need for long-term stability of the Mg temperature sensor. An initial calibration period after implantation is sufficient to define flow rates required to achieve a desired temperature. Placement of the temperature sensor at the distal end of the device ensures that it maintains contact with the nerve and provides a more accurate measure of nerve temperature than if located at the center, i.e., the coldest, location of the cooler.

FIG. 8 shows an in vitro benchtop setup for testing nerve cooling efficacy according to embodiment of the invention. Panel A: Schematic illustration of pump system and thermal bath. Panel B: Images of the experimental setup.

FIG. 9 shows a simulated temperature of the inlet flows according to embodiment of the invention. The temperature of the PFP (QPFP=300 μL/min) and N₂ (P_{N2}=34 kPa) reach approximately 37° C. within 80 mm after entering a 37° C. environment.

FIG. 10 shows a perfluoropentane condenser according to embodiment of the invention. Panel A: Schematic illustration of the experimental setup. Panel B: A coiled, 1 m long copper tube and a liquid trap condenses and collects gaseous PFP. N₂ and residual PFP vapor are vented to the atmosphere. Panel C: A cooling bath inside a vacuum insulated thermos provides means of condense the PFP. Panel D: Water ice, NaCl+water ice, and IPA+dry ice cooling baths enable collection of approximately 30, 70, and 90% of the PFP vapor, respectively.

FIG. 11 shows stability of two-phase cooling regimes according to embodiment of the invention. Two-phase evaporation flow stability experiments in ambient conditions reveals both stable and unstable flow regimes at PFP flow rates of (panel A) 8 μl/min, (panel B) 6 μl/min, (panel C) 32 μl/min, (panel D) 64 μl/min, and (panel E) 128 μl/min. Temperature fluctuations arise from instabilities in two-phase microfluidic flow, as described previously. A set of flow rate screening experiments reveals regimes of stable and unstable flow in ambient conditions. The average temperature value and temperature stability depend on both input flow rates and the ratio of the flow rates.

FIG. 12 shows corrected N₂ flows and calculated molar flow rates according to embodiment of the invention. Panel A: N₂ volumetric flow rate as measured with a rotameter. Panel B: N₂ volumetric flow rate after correction for super-atmospheric pressures. Panel C: N₂ molar flow rate. Panel D: Total (N₂+PFP) molar flow rate. Panel E: PFP molar fraction.

FIG. 13 shows an effect of nerve blood flow according to embodiment of the invention. Panel A: The experimental setup for evaluating the effect of nerve blood flow on cool efficacy. B Panel B: Temporal response data for the nerve temperature and nerve blood flow.

FIG. 14 shows a thermofluidic simulation according to embodiment of the invention. Panel A: Vapor-liquid equilibrium for PFP showing the saturated vapor relationship depends on the molar volume of gas Vₘ, the saturated concentration Cₛₐₜ(T), and the flow of N₂ (Q_{N2}). Panel B: Simulation workflow diagram.

FIG. 15 shows a simulation of the effect of blood flow on nerve temperature according to embodiment of the invention. Schematic illustrations of the simulation setup for quantifying the effect of nerve blood flow (panel A) radially and (panel B) longitudinally along the nerve. Panel C: The temperature distributions on the a-a cross section for Q_{blood}=0 and 50 μl/min. Panel D: The temperature distributions on the b-b cross section for Q_{blood}=0 and 50 μl/min.

FIG. 16 shows thermochromic hydrogel calibration setup according to embodiment of the invention. Panel A: Representative images of a thermochromic hydrogel during color calibration. Panel B: Illustrations of thermochromic hydrogel thickness.

FIG. 17 shows images of a microfluidic evaporative cooler embedded in a thermochromic hydrogel according to embodiment of the invention.

FIG. 18 shows images of a flat, uncurled microfluidic evaporative cooler embedded in a thermochromic hydrogel according to embodiment of the invention.

FIG. 19 shows temperature along the exterior of the microfluidic outlet channel according to embodiment of the invention. Panel A: Schematic illustration of the experimental setup. Panel B: Experimental results for the temperature of the exterior of the outlet channel (Q_{PFP}=300 μl/min, P_{N2}=34 kPa).

FIG. 20 shows the simulated temperature along the exterior of the exhaust channel with and without thermal management according to embodiment of the invention. Schematic illustration of the cross-section of (panel A) a design without air insulation (design A) and (panel B) a design with air insulation (design B). Temperature distribution at the interface of the device and surrounding tissue at 37° C. for (panel C) design A, (panel D) design B with h=0 Jim, (panel E) design B with h=200 Jim, and (panel F) design B with h=400 Jim. Panel G: The temperature distribution along the middle height (white dashed line) in the upper surface at the interface of the device and tissue. In all of these simulations, the gas properties are 12.3 kg m-3 for the density, 0.09 W m-1 K-1 for the thermal conductivity, 738 J kg-1 K-1 for the heat capacity, and 0.0178 cP for the viscosity. The exhaust flow rate is 100 mL/min and the initial exhaust gas temperature is 0° C.

FIG. 21 shows an experimental setup for acute animal trials according to embodiment of the invention. A soft curled cuff structure provides an intimate thermal and mechanical interface to the rat sciatic nerve without sutures and without mechanically induced damage. Hook electrodes placed proximal to a cooling cuff wrapped around a rat sciatic nerve serve as an experimental setup for acute in vivo electrophysiology studies.

FIG. 22 shows representative in vivo cooling profiles from acute trials according to embodiment of the invention. Rat sciatic nerve temperature response for (panel A) 300 μl/min, (panel B) 250 μl/min, and (panel C) 150 μl/min.

FIG. 23 shows bioresorbable and non-bioresorbable nerve coolers according to embodiment of the invention. Panel A: A bioresorbable platform consists of POC microfluidics and Mg temperature sensor, with non-bioresorbable interconnects comprising silicone tubes and stretchable Cu wires. Panel B: A non-bioresorbable platform consists of PDMS microfluidics and a negative temperature coefficient (NTC) temperature sensor, with non-bioresorbable interconnects comprising silicone tubes and stretchable Cu wires. Panel C: Silicone microfluidics, NTC temperature sensor, and stretchable Cu interconnect comprise a non-bioresorbable nerve cooler.

FIG. 24 shows device and connector design for awake, freely moving animal experiments according to embodiment of the invention. Panel A: Illustrated schematic device design for awake, freely moving animal experiments. Panel B: Illustration of the bioresorbable nerve cooler interfacing with the sciatic nerve of a rat. Panel C: Illustration of the subcutaneous routing path of the bioresorbable device and non-bioresorbable interconnects. A spared nerve injury (SNI) generates persistent sensitivity to touch (mechanical allodynia) and serves as a model for neuropathic pain. Panel D: Illustration of stretchable wires and silicone tubing that serve as interconnections to the headcap. Panel E: Stretchable fluidic and electronic interconnects and connectors. Panel F: Stretchable electrical interconnects formed from helical Cu wires embedded in silicone. Panel G: Fluidic/electronic terminal of the non-bioresorbable interconnects. Panel H: The terminal of the non-bioresorbable interconnects after mounting in the titanium headcap. Panel I: After connecting the external fluidic/electronic connector.

FIG. 25 shows a model for neuropathic pain according to embodiment of the invention. Higher mechanical nociceptive threshold on the SNI side as compared to the contralateral side over three weeks indicates persistent mechanical allodynia in control animals that only received the SNI (n=2).

FIG. 26 shows a histologic analysis according to embodiment of the invention. Panels A: H&E. Panel B: Toluidine blue-stained sections obtained from the interface of the nerve cuff and the sciatic nerve two and six months after implantation indicate close proximity of the cuff to the nerve, and a decrease in thickness of the POC provides evidence of ongoing degradation and bioresorption processed. Panel C: zoomed images indicate healthy axons. All samples exhibit mild, if any, evidence of axonal damage, ischemia, neutrophils, lymphocytes, eosinophils, or plasma cells which would characterize an acute or chronic inflammatory response (Table 1). A decrease in thickness of the outermost POC layer provides evidence of ongoing degradation and bioresorption processes, whereby the kinetics of bioresorption are governed by the materials chemistry and device geometry.

FIG. 27 shows a histologic analyses after 1-month implantations according to embodiment of the invention. Specimens taken from three animals with (panel A) H&E and (panel B) toluidine blue stains provide evidence of biocompatibility.

FIG. 28 shows a histologic analyses after 2-month implantations according to embodiment of the invention. Specimens taken from three animals with (panel A) H&E and (panel B) toluidine blue stains provide evidence of biocompatibility.

FIG. 29 shows a histologic analyses after 3-month implantations according to embodiment of the invention. Specimens taken from three animals with (panel A) H&E and (panel B) toluidine blue stains provide evidence of biocompatibility.

FIG. 30 shows a histologic analyses after 6-month implantations according to embodiment of the invention. Specimens taken from three animals with (panel) H&E and (panel B) toluidine blue stains provide evidence of biocompatibility.

FIG. 31 shows microfluidic channel dimensions according to embodiment of the invention. Panel A: Cross-sectional micrograph of the microfluidic interconnects. The microchannels have widths of 100 μm and 400 μm for transport of the PFP and N₂, respectively. PFP vapor vents via an outlet channel that is colinear to the two input microchannels and has a width of 400 μm. Panel B: Footprint of the serpentine evaporation chamber. The serpentine evaporation chamber consists of microfluidic channels with seven 180° turns, over a region with a total length, footprint, and volume of 75 mm, 76 mm², and 5.7 mm³, respectively.

FIG. 32 shows simulated effects of a biofluid-filled gap between the cuff and the nerve according to embodiment of the invention. Panel A: Schematic illustration of the footprint for the serpentine evaporation chamber. Panel B: Schematic illustration of the cross section of the simulation model. Panel D: 2D thermal simulation of temperature gradients across the cross section of the nerve with no gap, 100 μm, and 200 μm gap. Panel D: Average and max nerve temperature for 0, 100, and 200 μm biofluid filled gaps.

FIG. 33 shows an effect of curling extent according to embodiment of the invention. Panel A: Changing the length of the serpentine evaporation chamber from 3.49 to 5.85 mm of a 1.5 mm diameter nerve. Panel B: Increasing the coverage angle from 180° to 301° of a 1.5 mm diameter nerve. Panel C: 2D thermal simulation result of the temperature gradient across the nerve cross section. Panel D: As the wrapping ratio increases, the average temperature Tavg over the cross section, and the maximum temperature Tₘₐₓ of the nerve decreases significantly, and the temperature distribution becomes more uniform.

FIG. 34 shows a measurement of muscle force during nerve cooling according to embodiment of the invention. Intensity of muscular contraction in the extensor digitorum longus as measured by muscle force diminishes and is reversibly eliminated with reduced temperature of the sciatic nerve.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

The term “flexibility” or “bendability”, as used in the disclosure, refers to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. A used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and open or mesh geometries.

The term “bending stiffness” refers to a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

The terms “Young's modulus” and “modulus” are used interchangeably and refer to a mechanical property of a material, device or layer which refers to the ratio of stress to strain for a given substance. Young's modulus may be provided by the expression;

$$E = \frac{( s t r e s s )}{( strain )} = ( \frac{{L}_{0}}{Δ L} ) ( \frac{F}{A} ) ,$$

where E is Young's modulus, L₀ is the equilibrium length, AZ is the length change under the applied stress, F′ is the force applied and A is the area over which the force is applied. Young's modulus may also be expressed in terms of Lame constants via the equation:

$$E = \frac{μ ( 3 λ + 2 μ )}{λ + μ}$$

where λ and μ are Lame constants. High Young's modulus (or “high modulus”) and low Young's modulus (or “low modulus”) are relative descriptors of the magnitude of Young's modulus in a given material, layer or device. In some embodiments, a high Young's modulus is larger than a low Young's modulus, preferably 10 times larger for some applications, more preferably 100 times larger for other applications and even more preferably 1000 times larger for yet other applications. “Inhomogeneous Young's modulus” refers to a material having a Young's modulus that spatially varies (e.g., changes with surface location). A material having an inhomogeneous Young's modulus may optionally be described in terms of a “bulk” or “average” Young's modulus for the entire layer of material.

The term “elastomer”, as used in the disclosure, refers to a polymeric material which can be stretched or deformed and return to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers useful include, but are not limited to, thermoplastic elastomers, styrenic materials, olefenic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to, silicon containing polymers such as polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefenic materials, polyolefin, polyurethane thermoplastic elastomers, polyamides, synthetic rubbers, polyisobutylene, poly(styrene-butadiene-styrene), polyurethanes, polychloroprene and silicones. In one embodiment, a flexible polymer is a flexible elastomer.

The term “encapsulate” or “encapsulation”, as used in the disclosure, refers to the orientation of one structure such that it is at least partially, and in some cases completely, surrounded by one or more other structures. “Partially encapsulated” refers to the orientation of one structure such that it is partially surrounded by one or more other structures. “Completely encapsulated” refers to the orientation of one structure such that it is completely surrounded by one or more other structures. The invention includes devices having partially or completely encapsulated electronic devices, device components and/or inorganic semiconductor components.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Implantable devices capable of targeted and reversible blocking of peripheral nerve activity may provide compelling alternatives to opioids for treating pain. Local cooling represents an attractive means for on-demand elimination of pain signals, but traditional technologies are limited by rigid, bulky form factors, imprecise cooling, and requirements for extraction surgeries.

This invention introduces classes of soft, bioresorbable, microfluidic devices that enable delivery of focused, minimally invasive cooling power at arbitrary depths in living tissues with real-time temperature feedback control. Construction with water-soluble, biocompatible materials leads to dissolution and bioresorption as a mechanism to eliminate unnecessary device load and risk to the patient without additional surgeries. Multiweek in vivo trials demonstrate the ability to rapidly and precisely cool peripheral nerves to provide local, on-demand analgesia in rat models for neuropathic pain for up to three weeks.

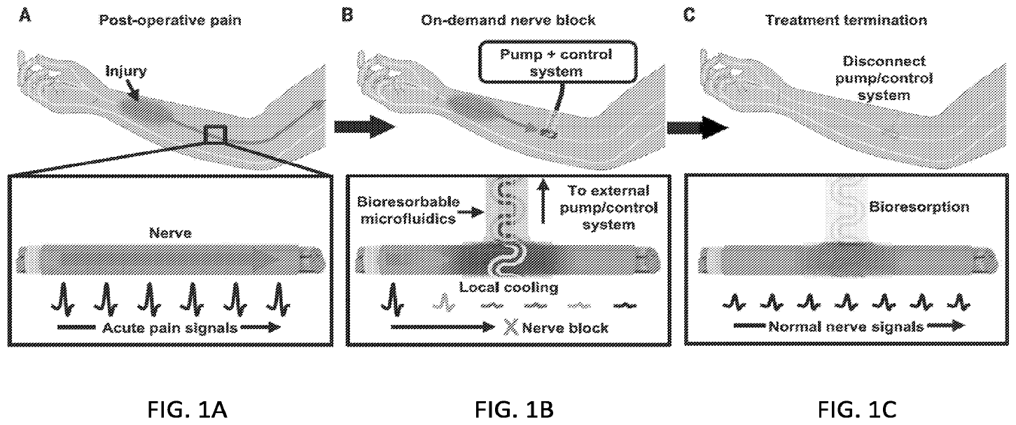

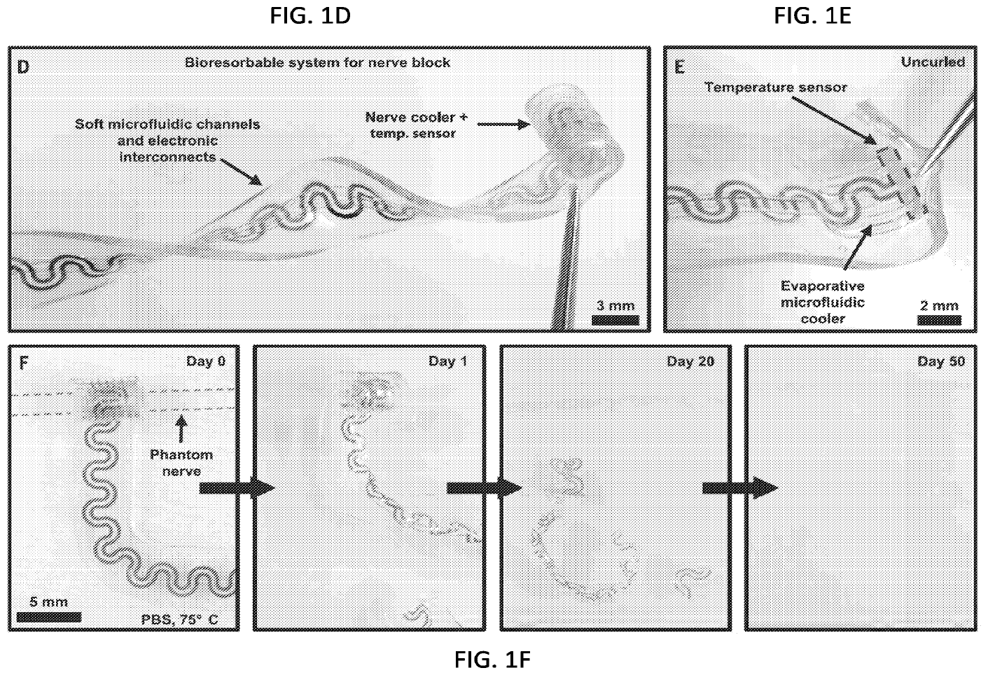

An implantable device that provides on-demand local analgesia over a defined timeline followed by subsequent dissolution and bioresorption would represent a qualitative advance in pain management techniques. An important envisioned use case is in the non-opioid management of post-operative acute pain signals in peripheral nerves (FIG. 1A). In this scheme, implantation of a bioresorbable cooler around the nerve that innervates damaged tissue enables reversible elimination of neural activity and pain signals via the focused application of cooling power (FIG. 1B). Construction from water soluble materials leads naturally to dissolution of the cooling system after the completion of the healing process and obviates the need for an extraction surgery (FIG. 1C), in a manner conceptually to that of other recently reported classes of bioresorbable sensors, therapeutic and delivery systems to monitor wound healing or recovery processes.

Among other things, the disclosure includes (i) designs for microfluidic channels and serpentine evaporation chambers optimized for localized evaporative cooling, with millimeter-scale spatial precision; (ii) soft, bioresorbable material structures that combine microfluidic and electronic functionality; (iii) 3D thermofluidic models of microfluidic evaporative cooling; and (iv) demonstrations of longitudinal cooling of peripheral nerves, and consequent nerve block, in awake, freely moving animals. The soft physical formats allow for long-lived, biocompatible, and intimate mechanical and thermal interfaces to peripheral nerves. These features, together with capabilities in spatiotemporally precise, large-amplitude cooling with integrated sensing capabilities for closed-loop control qualitatively differentiate this work from previous implantable cooling systems. The results may have important implications for the treatment of pain, where engineering approaches offer the potential to bypass the need for pharmaceutical interventions across a range of important clinical use cases.

Specifically, in one aspect, the invention relates to a device for reversibly blocking activities of a target region of a subject comprising a microfluidic system configured to route a fluid around the target region to change a local temperature of the target region so as to reversibly block activities of the target region.

In some embodiments, the microfluidic system is operably in communication with the target region.

In some embodiments, the microfluidic system utilizes a liquid to gas phase transition as a cooling mechanism to change the local temperature of the target region.

In some embodiments, the microfluidic system comprises at least one fluidic chamber formed in a microfluidic layer.

In some embodiments, the at least one fluidic chamber has a length, a footprint and a volume, wherein the length defines a coverage angle of the microfluidic system when wrapping around the target region.

In some embodiments, a cooled area in the target region is confined predominately to a surface directly associated with the at least one fluidic chamber.

In some embodiments, the microfluidic system further comprises transcutaneous colinear interconnects that deliver the fluid to the at least one fluidic chamber in a completely sealed system that provides fluidic access at the ends.

In some embodiments, the microfluidic system further comprises at least first and second input channels and at least one output channel fluidically connected to the at least one fluidic chamber.

In some embodiments, the at least one output channel is colinear to the at least first and second input channels.

In some embodiments, the first and second input channels have widths in ranges of about 50-150 μm and about 200-600 μm, respectively, and the output channel has a width in a range of about 200-600 μm.

In some embodiments, the fluid comprises a coolant and a dry gas being operably transported into the at least one fluidic chamber via the first and second input channels, respectively.

In some embodiments, the microfluidic system is configured such that a simultaneous initiation of the coolant and the dry gas flows into the at least one fluidic chamber prompts evaporation of the coolant at a microfluidic junction between the first and second input channels of the coolant and the dry gas and along the at least one fluidic chamber.

In some embodiments, the at least one fluidic chamber comprises at least one serpentine microfluidic channel formed with a plurality of U-shaped turns over a region in the microfluidic layer.

In some embodiments, the at least one serpentine microfluidic channel operably routes a volume of the coolant to the target region where a flow of the dry gas triggers local and fully contained evaporation of the coolant.

In some embodiments, mass flow rates of the coolant and the dry gas, and the length, the footprint and the volume of the at least one fluidic chamber determine magnitude and localization of the cooling effect.

In some embodiments, the coolant is fluorocarbons, and wherein the dry gas comprises any dry gas including N₂, CO₂, argon, or mixtures thereof.

In some embodiments, the coolant is a bioinert coolant including perfluoropentane (PFP).

In some embodiments, the coolant is a non-bioinert coolant including diethyl ether.

In some embodiments, the microfluidic system further comprises at least one first pump in fluidic communication with the at least first and second input channels for delivering the fluid to the at least one fluidic chamber to change the local temperature of the target region.

T In some embodiments, the microfluidic system further comprises at least one second pump in fluidic communication with the at least one output channel for withdrawing the fluid from the at least one fluidic chamber.

In some embodiments, the device is configured such that a phase change prompts a temperature of the device in a planar, uncurled configuration to drop to about −20° C. within about 2 min or less after initializing flow in ambient, room temperature conditions.

In some embodiments, the microfluidic system is formed of a bioresorbable elastomer.

In some embodiments, the bioresorbable elastomer is selected to have an elastic modulus that is compatible with the target region, with a controllable rate of degradation via surface erosion.

In some embodiments, the elastic modulus is in a range of about 10 kPa and 1 GPa.

In some embodiments, the bioresorbable elastomer comprises biopolymers, synthetic polymers, proteins, polysaccharides, poly(octanediol citrate) (POC), polydioxanone, poly(lactic-co-glycolic acid) (PLGA), polylactic acid (PLA), collagen, chitosan, poly(glycerol sebacate), poly(lactic-co-glycolic acid), poly(caprolactone), poly(octamethylene maleate (anhydride) citrate, poly(itaconate-co-citrate-co-octanediol).

In some embodiments, the microfluidic system is formed of a non-bioresorbable elastomer.

In some embodiments, the non-bioresorbable elastomer comprises thermoplastic elastomers, styrenic materials, olefenic materials, polyolefin, polyurethane thermoplastic elastomers, polyamides, synthetic rubbers, polydimethylsiloxane, polybutadiene, polyisobutylene, poly(styrene-butadiene-styrene), polyurethanes, polychloroprene, silicones, or a combination thereof.

In some embodiments, the device further comprises an electronic system coupled with the microfluidic system for providing a real-time feedback.

In some embodiments, the electronic system comprises at least one sensor configured to detect a temperature of the target region for providing a temperature feedback.

In some embodiments, the at least one sensor is a flexible temperature sensor.

In some embodiments, the at least one sensor is a bioresorbable, resistance-based temperature sensor.

In some embodiments, the at least one sensor comprise a magnesium resistance-based temperature sensor.

In some embodiments, the magnesium resistance-based temperature sensor comprises a serpentine magnesium trace.

In some embodiments, the at least one sensor comprises a layer of magnesium (Mg) encapsulated with silicon dioxide (SiO₂) and supported by a cellulose acetate substrate.

In some embodiments, the at least one sensor comprises a multilayered stack of SiO₂/Mg/SiO₂ deposited on a cellulose acetate substrate.

In some embodiments, the at least one sensor is encapsulated by two layers of the bioresorbable elastomer of POC.

In some embodiments, the electronic system operably lies coplanar with the microfluidic system, with the at least one temperature sensor at the distal end of the device.

In some embodiments, the target region comprises soft tissue structures including a peripheral nerve.

In some embodiments, the electronic and microfluidic systems terminate in a cuff structure with a diameter matched to the peripheral nerve, to provide an intimate mechanical and thermal interface to the nerve, without the need for sutures.

In some embodiments, the device further comprises a collection member coupled to the microfluidic system for condensing and collecting gaseous coolant.

In some embodiments, the collection member comprises a condenser connected to the at least one output channel of the microfluidic system, and a liquid trap connected to the condenser.

In some embodiments, the collection member further comprises a pressure meter connected at a point between the output channel of the microfluidic system and an inlet to the condenser for measuring backpressure of the condenser.

In some embodiments, the device is configured to allow for long-lived, biocompatible, and intimate mechanical and thermal interfaces to peripheral nerves.

In some embodiments, the device is capable of spatiotemporally precise, large-amplitude cooling with integrated sensing capabilities for closed-loop control.

In some embodiments, the device is configured to deliver focused, minimally invasive cooling power at arbitrary depths in living tissues with real-time temperature feedback control.

In some embodiments, the device is usable for a treatment of pain, without a need for pharmaceutical interventions.

In some embodiments, the device is implantable device.

In some embodiments, the device is a hybrid wearable/implantable device

In some embodiments, the device is biocompatible.

In some embodiments, the device is constructed entirely with water-soluble constituent materials that are controllably dissolvable in biofluids and are subsequently bioresorbable therein.

In another aspect, the invention relates to a method for reversibly blocking activities of a target region of a subject, comprising routing a fluid around the target region to change a local temperature of the target region so as to reversibly block activities of the target region.

In some embodiments, said the fluid around the target region is performed by a microfluidic system that utilizes a liquid to gas phase transition as a cooling mechanism to change the local temperature of the target region.

In some embodiments, the microfluidic system comprises at least one fluidic chamber formed in a microfluidic layer.

In some embodiments, the microfluidic system further comprises at least first and second input channels and at least one output channel fluidically connected to the at least one fluidic chamber.

In some embodiments, the fluid comprises a coolant and a dry gas being operably transported into the at least one fluidic chamber via the first and second input channels, respectively.

In some embodiments, the microfluidic system is configured such that a simultaneous initiation of the coolant and the dry gas flows into the at least one fluidic chamber prompts evaporation of the coolant at a microfluidic junction between the first and second input channels of the coolant and the dry gas and along the at least one fluidic chamber.

In some embodiments, the at least one fluidic chamber comprises at least one serpentine microfluidic channel formed with a plurality of U-shaped turns over a region in the microfluidic layer.

In some embodiments, the at least one serpentine microfluidic channel operably routes a volume of the coolant to the target region where a flow of the dry gas triggers local and fully contained evaporation of the coolant.

In some embodiments, the method further comprises measuring a temperature of the target region to provide a real-time feedback.

In some embodiments, said measuring the temperature of the target region is performed by a flexible temperature sensor.

In some embodiments, the flexible temperature sensor is bonded to the microfluidic system.

In yet another aspect, the invention relates to a method for fabricating a device for reversibly blocking activities of a target region of a subject, comprising fabricating a microfluidic system for routing a fluid around the target region to change a local temperature of the target region; and fabricating an electronic system coupled with the microfluidic system for providing a real-time feedback.

In some embodiments, said fabricating the microfluidic system comprises providing a patterned substrate; coating a sacrificial layer on the patterned substrate; casting a prepolymer on the coated sacrificial layer and subsequently curing the casted prepolymer to form a polymer layer; and removing the sacrificial layer from the polymer layer to define at least one fluidic chamber with at least one at least one serpentine microfluidic channel.

In some embodiments, the sacrificial layer is formed of poly(acrylic acid) (PAA).

In some embodiments, the prepolymer is a poly(octanediol citrate) (POC) prepolymer.

In some embodiments, said fabricating the electronic system comprises sequentially depositing SiO₂, Mg and SiO₂ onto cellulose acetate to form a temperature sensor layer; and encapsulating the temperature sensor layer with top and bottom layers of POC.

In some embodiments, the method further comprises bonding the electronic system to the microfluidic system.

The embodiments disclosed herein establish the foundations in engineering science for this class of technology. Specifically, the work introduces concepts and device designs for soft, bioresorbable peripheral nerve cooling and temperature sensing units engineered to reversibly block pain signals with a targeted cooling stimulus, over a finite time period matched to patient needs. Key advances are in the use of a liquid to gas phase transition as a cooling mechanism and in soft microfluidic structures as a bioresorbable delivery platform. Here, elastomeric microfluidic channels route microliter volumes of a bioinert coolant to peripheral nerves where flow of a dry gas triggers local and fully contained evaporation of the coolant. The enthalpy, or latent heat, of evaporation generates the cooling effect. This approach presents significant advantages for rapidly creating large-amplitude, localized cooling effects with relaxed fluid delivery requirements as compared to traditional techniques that rely on forced liquid convection. Specifically, the cooling power associated with this scheme can exceed that of forced convection by one to two orders of magnitude. In addition, the use of a compressible working fluid lowers the operating pressures and the occurrence of transient pressure spikes, by comparison to systems that use forced convection. These unusual operating parameters enable the use of thin, soft bioresorbable materials for microfluidic delivery structures that can also integrate with flexible electronic sensors for measuring nerve temperature. As a consequence, this type of evaporative cooling, previously established in the context of microelectronic thermal management and lab-on-chip systems, can now be exploited as implantable, bioresorbable interfaces to small, soft tissue structures such as peripheral nerves.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example

### Soft, Bioresorbable Coolers for Reversible Conduction Block of Peripheral Nerves

The high therapeutic efficacy of opioids has led to their widespread use despite increasing rates of addiction and opioid-related deaths due to overdose. The increasing societal burden caused by opiate misuse motivates the development of localized, nonopioid, and nonaddictive pain-management techniques. Miniaturized implantable devices that eliminate pain signals locally in peripheral nerves suggest a potential role for engineering-based treatments that avoid side effects associated with opioids and other analgesics. The controlled input of electrical, pharmacological, optical, mechanical, or thermal stimuli to neural tissue can lead to local and reversible neural blocking.

Of particular interest for the study herein is that temporal measures of metabolic, electrogenic, and ionic activity in neural tissue all exhibit a negative temperature dependence. Local cooling of peripheral nerves decreases conduction velocity and signal amplitude of neural activity. Blocking of transmission of compound action potentials in mammalian nerves typically occurs below 15° C., but this threshold can be temporarily increased to near room temperature by a brief heating period preceding the cooling period. Cooling applied to peripheral nerves is a promising approach for blocking pain signals because it is nonaddictive, is rapidly reversible, can be applied locally, avoids any onset response, and allows for simultaneous electrical interrogation of the blocked nerve. Analgesic nerve cooling requires spatiotemporally precise control of temperature to maximize desired outcomes and to minimize the chance of cooling-induced tissue damage. Current approaches for nerve cooling rely on rigid, bulky systems that prevent the use of local cooling as a practical approach for peripheral nerve pain management.

An implantable device that provides on-demand local analgesia over a defined timeline followed by subsequent dissolution and bioresorption would represent a qualitative advance in pain-management techniques. We specifically envision clinical use cases for non-opioid management of postoperative acute pain signals in peripheral nerves where (i) aberrant neural signals are well defined in select anatomical regions, (ii) nerves carrying aberrant neural signals are already isolated, and (iii) a need for opioid therapy exists after operation (FIG. 1A). Pain management after amputations, nerve grafts, or spinal decompression surgeries represent examples. Here, the relevant nerves are already isolated and identified; thus, the application of the cuff would be straightforward to integrate into the clinical workflow. In this scheme, implantation of a bioresorbable cooler around the nerve that innervates damaged tissue enables reversible elimination of neural activity and pain signals through the focused application of cooling power (FIG. 1B). Construction from water-soluble materials leads naturally to dissolution of the cooling system after the completion of the healing process and obviates the need for an extraction surgery (FIG. 1C), in a manner conceptually similar to that of other recently reported classes of bioresorbable sensors and therapeutic systems to monitor and accelerate wound healing or recovery processes.

In this exemplary study, we disclose soft, bioresorbable, microfluidic devices that enable delivery of focused, minimally invasive cooling power at arbitrary depths in living tissues with real-time temperature feedback control. The devices are engineered to reversibly block pain signals with a targeted cooling stimulus over a finite time period matched to patient needs. Multiweek in vivo trials of the devices in animals demonstrate the ability to rapidly and precisely cool peripheral nerves to provide local, on-demand analgesia in rat models for neuropathic pain. In addition, the devices are constructed with water-soluble, biocompatible materials leading to dissolution and bioresorption as a mechanism to eliminate unnecessary device load and risk to the patient without additional surgeries.

### Materials and Methods

Microfluidic channel layer mold fabrication: Exposing a 10 μm thick layer of negative photoresist (KMPR 1010; spun coat at 3000 RPM and 1000 RPM/s for 30 s and soft baked at 110° C. for 5 min) on a silicon wafer to ultraviolet light (420 mJ/cm²) through a chrome mask followed by soft baking (110° C. for 5 min) and developing (AZ 917MIF, 3 min) produced patterns in the resist. A deep reactive ion etching (DRIE) Bosch process created trenches in the exposed regions of the silicon wafer to a depth of 250 μm (STS Pegasus ICP-DRIE, SPTS Technologies Ltd.).

Poly (octanediol citrate) (POC) prepolymer synthesis: The steps for synthesis followed a previously reported process. Briefly, adding equimolar amounts of citric acid (Fisher Scientific) and 1,8-octanediol (Alfa Aesar) to a 500 ml round bottom flask prepared the system for esterification. A stream of N₂ (500 ml/min) provided an inert atmosphere. Submerging the flask in a 140° C. silicone oil bath under constant magnetic stirring melted the reactants after ˜20 min. Reacting for an additional 30 min produced a viscous, clear prepolymer which was stored in sealed vials (21° C., 40% RH).

POC layer fabrication: Submersion of patterned and flat silicon wafers in an organic stripping bath (Cyantek Nanostrip) at 70° C. and subsequent plasma etching (March RIE, 200 W, 200 mT, 30 s) yielded clean, oxidized silicon surfaces. Spin coating (1000 rpm, 45 s) a 5 wt % poly(acrylic acid) (PAA) Sigma Aldrich) solution (neutralized with sodium hydroxide to a pH of 7.5) and subsequent heating at 110° C. for 2 min produced a water-soluble sacrificial layer. Casting 4.0 g of POC prepolymer on the 4″ PAA-coated microfluidic channel layer yielded POC films ˜425 μm thick after curing with 250 μm deep channels. Spin coating a solution of 90 wt % POC prepolymer in acetone at 600 rpm for 30 sec on flat silicon wafers yielded flat layers 120 μm thick after curing. Heating POC prepolymer layers under vacuum (120° C., 27 inHg) for 4 hours produced partially cured, tacky POC films. Submersion in 80° C. deionized water for 45 min dissolved the PAA sacrificial layer. Drying the top surface of the POC with a stream of N₂ and laminating a sheet of water-soluble tape (Aquasol) provided support for transferring the POC to a carrier layer of silicone rubber. Rinsing under deionized water removed the water-soluble tape. Heating under vacuum (120° C., 27 inHg) for 30 min removed residual water from the partially cured POC films while supported by the silicone carrier.

Temperature sensor fabrication: Sequential electron beam depositions (AJA International Inc., MA, USA) of SiO₂/Mg/SiO₂ (100/300/100 nm) onto cellulose acetate (50 μm, Goodfellow Corporation, PA, USA) yielded a Mg layer insulated with SiO₂. Laser machining (LPKF Protolaser R) defined both the Mg temperature sensor and the outer border of the cellulose acetate. Exposing two partially cured POC layers (120 μm thick) to UV ozone (Jelight 144AX) for 8 min yielded tacky surfaces and prepared the POC for bonding. The temperature sensor layer was laminated to a freestanding layer of POC (120 μm) and the second POC layer (120 μm) was laminated on top. Applying pressure with binder clips with silicone rubber layers on either side of the POC-laminate structure and heating for 30 min under vacuum (120° C., 27 inHg) permanently bonded the POC encapsulated temperature sensor.

Temperature sensor characterization: Submersion of the temperature sensor in a temperature-controlled water bath provided a means of quantifying temperature sensitivity. Manual addition of ice and warm water provided means of setting the bath temperature between 0-37° C. A type-k thermocouple provided the reference value of the bath. Benchtop longevity of POC encapsulated devices were evaluated in a 37° C. bath of PBS (pH 7.4). Resistance values were recorded with a PC controlled logger (Neulog NUL-259).

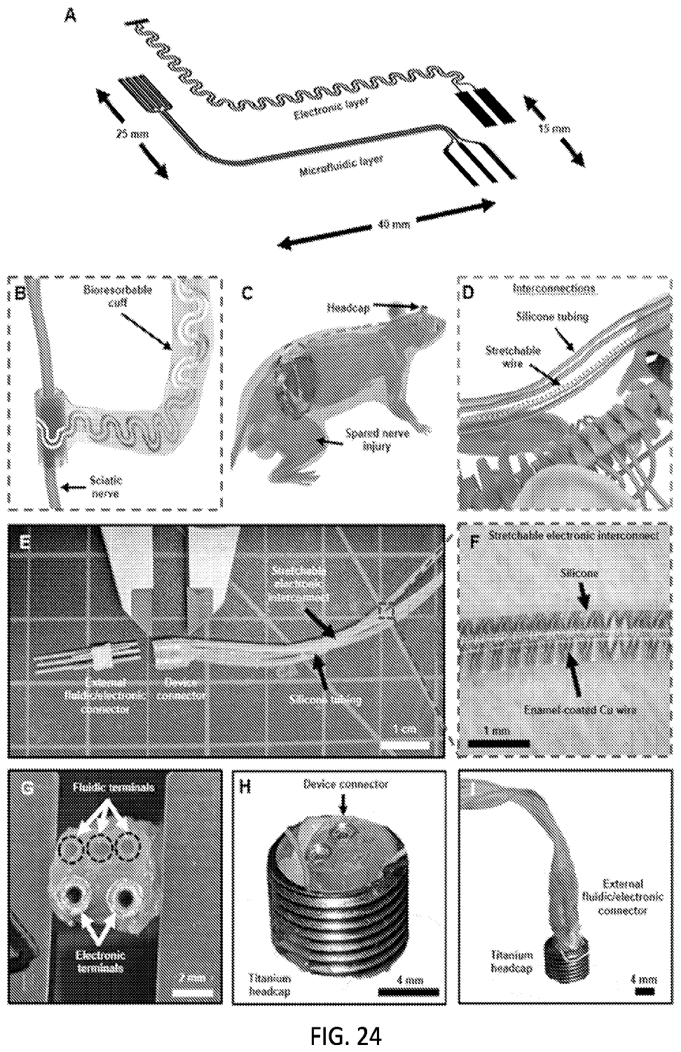

Stretchable interconnects and fluidic/electronics connector: Custom stretchable interconnects and connectors provided fluidic and electronic connectivity (FIG. 24). Enamel coated Cu wire (38 AWG) coiled around a 0.75 mm mandrel with a pitch of 0.6 mm formed the stretchable electronic interconnects. Transferring the coiled Cu wire from the mandrel into a tube with an inner diameter of 0.9 mm prepared the coil for embedding in silicone. Injection of silicone (Sylgard 184, 10:1 ratio) and subsequent curing completed the stretchable interconnect. Scoring the tubing allowed extraction of the stretchable interconnect. Receptacle connectors (Digi-key, 835-43-002-40-030001) soldered to one end of the Cu wires served as a connector to the external temperature logger. Header pins (Digi-key, ED3864-02-ND) connected to flexible wires (Calmont, 36 AWG) served as a detachable electrical connection to the device. Stainless steel (OD=0.9 mm, ID=0.6 mm) and silicone tubing (McMaster, OD=1.2 mm, ID=0.64 mm) served as a detachable fluidic connection to the device. Fluidic and electrical terminals were integrated into a single plug using silicone adhesive (Smooth-On Silpoxy).

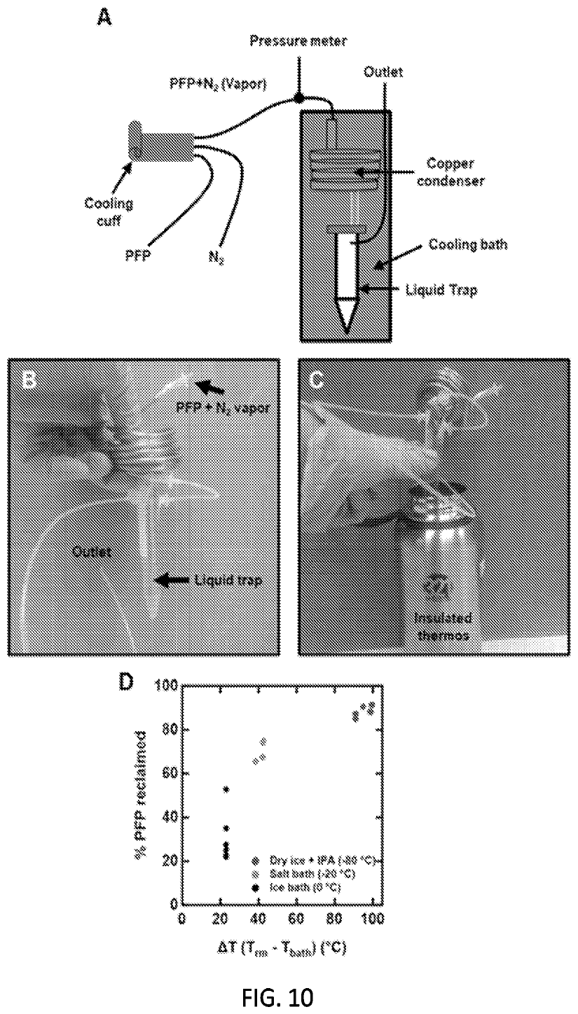

Hybrid microfluidic-electronic assembly: Exposing the POC microfluidic layer and POC encapsulated temperature sensor layer to UV ozone (Jelight 144AX) for 8 min yielded tacky surfaces and prepared the POC for bonding. Laminating the flat and patterned POC layers and subsequently heating under vacuum for 30 min (120° C., 27 inHg) permanently bonded the POC layers. Manually curling around a 1 mm stainless steel tube and curing for an additional 24 hrs (120° C., 27 inHg) yielded a fully cured, curled device. Silver epoxy (MG Chemicals) provided electrical connectivity between stretchable Cu interconnects and the Mg pads of the bioresorbable temperature sensor. An overlay of marine epoxy (Loctite) provided mechanical Experimental setup for microfluidic evaporative cooling: Silicone tubing (McMaster, 1.2 mm OD, 0.64 mm ID, 50 cm long) inserted into the perfluoropentane inlet, N₂ inlet, and outlet of the POC device and fixed in place with adhesive (Smooth-On Silpoxy) provided fluidic connections. Submerging lengths of 150 mm or more of inlet tubing in the 37° C. bath eliminates any cooling contributions that arise from PFP and/or N₂ at the temperature of the ambient environment. A syringe pump (New Era NE-300) provided means of volumetric flow control of perfluoropentane (Fluoromed Specialty Chemicals). Vertical orientation of the syringe pump during experiments prevented collection of PFP vapor bubbles in the syringe. Pressure-regulated N₂ was supplied from a central laboratory source. The N₂ flow was supplied in a pressure driven support. mode to mitigate transient pressure spikes resulting from pressure instabilities in the multi-phase flow. Volumetric N₂ flow rate was measured via a variable area flow rotameter (Dwyer Instruments) and subsequently corrected for superatmospheric pressures. The flow from the vapor outlet was fed to a custom condenser and liquid collector in a −80° C. bath (dry ice and isopropanol) inside a vacuum insulated thermos (FIG. 10).

Benchtop hydrogel studies of cooling efficacy: A commercial sous vide cooker maintained a water bath at 37+0.5° C. Agarose hydrogels (0.5 wt %, Sigma Aldrich) served as a tissue mimic. A type-k thermocouple (Omega) embedded in a thermally conductive silicone cylinder (Sylgard 170, 1.5 mm diameter) served as an instrumented phantom nerve.

Perfluoropentane condenser tests: A custom condenser and liquid trap formed from a vacuum insulated thermos, copper tubing (McMaster OD=3.175 mm ID=1.549 mm), and Teflon® tubing (Cole Parmer, OD=1.6 mm, ID=1 mm) recaptured PFP vapor. Luer lock fittings and marine epoxy provided connections to the nerve cooler and condenser. A pressure meter (Neulog NUL 210) connected at a point between the outlet of the cuff and inlet to the condenser measured the backpressure of the condenser. Cooling baths formed from water ice, NaCl and water ice, and dry ice and isopropanol provided bath temperatures of 0, −20, and −80° C. Evaporative tests were run at 200 μl/min, P_{N2}=34 kPA for 15 min for a total liquid PFP volume of 3 ml. Visual inspection of the 5 ml conical tube with graduated volumetric markings yielded the volume of the PFP condensate. Tests were run in sets of five.

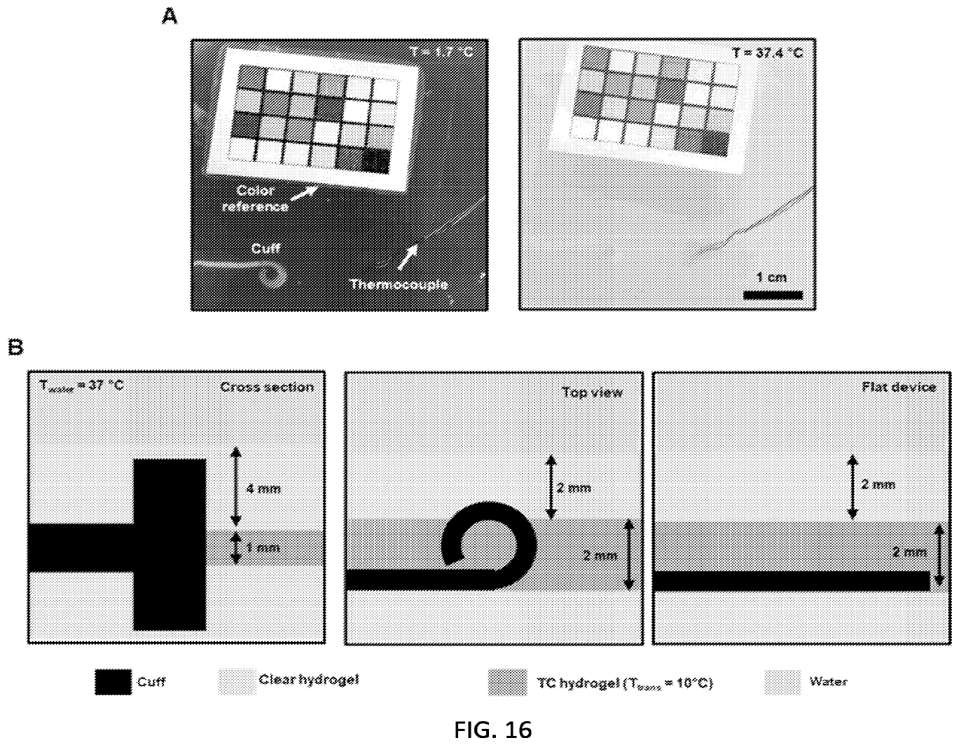

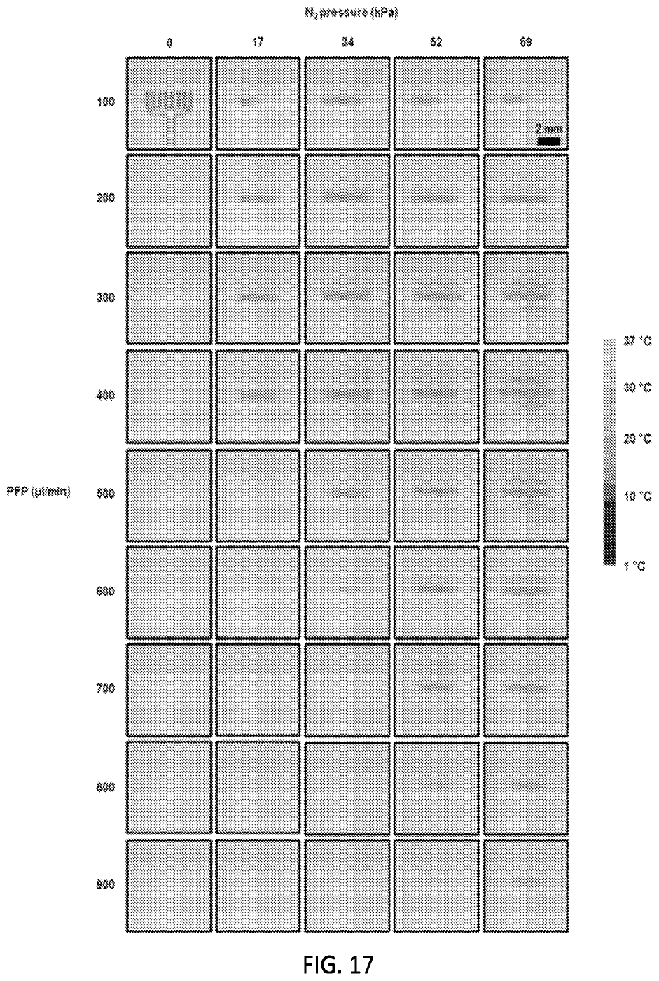

Cooling localization experiments: A hydrogel composite formed from 0.5 wt % agarose (Sigma Aldrich) and 0.25 wt % of thermochromic leuco dye (10° C. activated thermochromic pigment, LCR Hallcrest) served as a thermochromic tissue mimic. A 1- or 2-mm thick layer of thermochromic hydrogel oriented perpendicular to the viewing direction and embedded within a clear hydrogel facilitates visualization of the 10° C. thermocline. Neat 0.5 wt % agarose served as the clear hydrogel. Reference images for forming the temperature calibration were captured while modulating the bath temperature from 1-37° C. using an ice bath and the sous vide cooker. Images taken with a DSLR camera (Canon EOS Rebel T6i) and auxiliary lighting were color balanced using a color reference palette embedded in the hydrogel (FIG. 16, panel A). Evaporative cooling flow conditions: PFP: 300 μl/min, N₂: 34 kPa.

Effects of blood flow on nerve cooling: A rubber tube (1.5 mm OD, 0.5 mm ID, Tygon Microbore) served as a phantom nerve with approximate dimensions and thermal properties of a rat sciatic nerve. 37° C. saline pumped through the phantom nerve at physiologically relevant flow rates (rat sciatic nerve, 0-50 μl/min) via a syringe pump (New Era NE300) served as a mimic for nerve blood flow. Evaporative cooling flow conditions: PFP: 300 μl/min, N₂: 34 kPa. The nerve temperature was allowed to equilibrate for 120 sec before recording the temperature. Data points and error bars represent means±S.E.M. of three trials. Standard deviation was calculated from three experiments using the same device.

In vitro benchtop studies of microfluidic longevity: POC nerve coolers fitted to a silicone phantom nerve instrumented with a type-k thermocouple (Omega) and submerged is 37° C. PBS (pH 7.4) for 22 days. Daily application of flow conditions for 3 min (PFP: 400 μl/min, N₂: 34 kPa) provide means of determining the functional lifetime.

Accelerated degradation tests: A 75° C. PBS (pH 7.4) bath served as an accelerated aging environment for bioresorbable microfluidic coolers and was changed every two days.

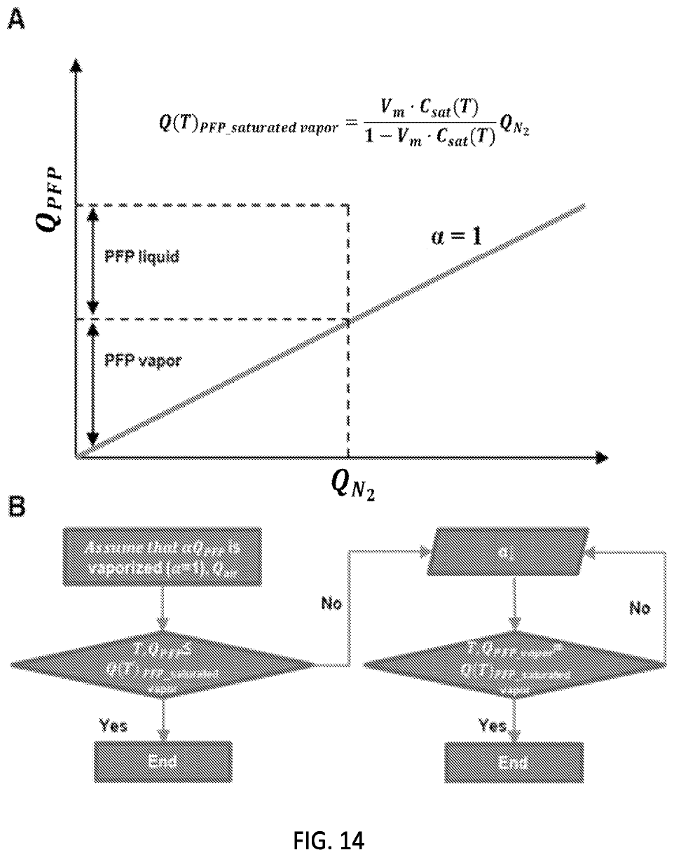

Thermofluidic simulations: A thermofluidic model was developed to numerically determine the temperature change resulting from evaporation in a microchannel. Liquid PFP and N₂ are introduced at flow rates Q_{PFP} and Q_{N2} in two colinear inlet channels before reaching a junction and subsequent evaporation chamber where, for a given temperature T, the PFP vapor becomes saturated in the N₂ when there is sufficient PFP (for given N₂) (FIG. 14A). The flow rate of PFP saturated vapor is given by

$${Q ( T )}_{PFP \_ saturated vapor} = \frac{{V}_{m} \cdot {C}_{s a t} ( T )}{1 - {V}_{m} \cdot {C}_{s a t} ( T )} {Q}_{{N}_{2}} \qquad ( Eq . l )$$

where

$${C}_{s a t} ( T ) = \frac{{P}_{s a t} ( T )}{R T}$$

is the saturated concentration of PFP that depends on the vapor pressure Pₛₐₜ(T), R is the ideal gas constant, and Vₘ is the molar volume of N₂ gas that depends on the temperature and pressure. The vaporization ratio is given by

$$α = \frac{{Q ( T )}_{PFP \_ saturated vapor}}{{Q}_{PFP}} \qquad ( Eq . 2 )$$

where α=1 indicates that the PFP is completely vaporized at the calculated T. At a given T, α is modified accordingly to reach the saturated vapor region as shown in FIG. 14B if the Q_{PFP} is larger than the saturated vapor (i.e., Q_{PFP}≥Q(T)_{PFP_saturated vapor}). Convergence tests of the mesh size were performed to ensure accuracy. The thermal properties used in the simulation are listed in Table 1.

TABLE 1
Material parameters used in the thermofluidic simulation (at 273 K and 1
standard atmosphere)

| | | Thermal | | | | |
|---|---|---|---|---|---|---|
| | Density | conductivity | Heat capacity | Viscosity | Molar mass | Standard state |
| | ρ | λ | C | v | M | enthalpy |
| | (kg m⁻³) | (W m⁻¹ K⁻¹) | (J kg⁻¹ K⁻¹) | (cP) | (kg kmol⁻¹) | (J kgmol⁻¹) |
| PFP | 1630 | 0.5 | 653 | 0.2440 | 288 | −25.74e8 |
| Liquid | | | | | | |
| PFP Gas | 12.3 | 0.09 | 738 | 0.0178 | 288 | −25.43e8 |
| N₂ | 1.138 | 0.0242 | 1040.67 | 0.0166 | 28 | 0 |
| Water | 998 | 0.6 | 4182 | 1.0030 | — | — |
| TC | 998 | 0.625 | 4120 | — | — | — |
| Hydrogel | | | | | | |
| PDMS | 1150 | 0.18 | 1206 | — | — | — |

Evoked muscle force measurement: All animal procedures were performed in strict accordance with the Animal Studies Committee and the Division of Comparative Medicine at Washington University School of Medicine (protocol #20180103). All procedures were performed on adult (200-250 g) male Sprague-Dawley rats. All acute trials were performed with a single animal. Surgery was performed under isoflurane (2%) anesthesia. Sciatic nerve function was terminally evaluated by examining force production in musculature on electrical stimulation of the sciatic nerve. Following surgical exposure, the distal tendon of the extensor digitorum longus (EDL) muscle was fashioned into a loop and secured to a stainless-steel S-hook at the musculotendinous junction using 6-0 nylon suture. Animals were subsequently placed in a designed functional assessment station (FAST System, version 2.0; Red Rock Laboratories) wherein the right leg was immobilized at the femoral condyles. The stainless steel S-hook was then connected to a 10 N thin-film load cell (S100; Strain Measurement Devices) supported on an adjustable mount. Cathodic, monophasic electrical impulses (duration=50 μs, frequency=single pulse, amplitude=1 mA) were delivered to the sciatic nerve proximal to the implanted device. Resulting force production in the isolated EDL muscle was transduced via the load cell and recorded on a desktop PC equipped with data acquisition software (version 2.0; Red Rock Laboratories). Thermal conduction and metabolic processes of the animal prompt nerve rewarming in all cases.

Evoked neuromuscular response: Cathodic, monophasic electrical impulses (duration=50 μs, frequency=single pulse, amplitude=1 mA) were delivered to the sciatic nerve proximal to the implanted device. EMG responses were collected from the tibialis anterior muscle using Red Rock Laboratories data acquisition software (version 2.0) and analyzed using the MATLAB software, version 2009B (MathWorks). Raw EMG data were rectified to a single polarity.

Evoked compound nerve action potential measurement: Sciatic nerve function was assessed in situ by examining compound neural action potential (CNAP) conduction across cooled nerves. Cathodic, monophasic electrical impulses (duration=50 μs, frequency=single pulse, amplitude=1 mA) were generated by a single-channel isolated pulse stimulator (Model 2100, A-M Systems Inc., Carlsborg, WA) and delivered to the sciatic nerve proximal to the implanted device via bipolar silver wire electrodes (4 mil, California Fine Wire, Grover Beach, CA). Resulting CNAPs were then differentially recorded distal to the implanted device at the peroneal nerve using similar bipolar silver wire electrodes. Measured signals were bandpass filtered (LP=1 Hz, HP=5 kHz, notch=60 Hz) and amplified (gain=1000×) using a two-channel microelectrode AC amplifier (Model 1800, A-M Systems Inc., Carlsborg, WA) before being recorded on a desktop PC (Dell Computer Corp., Austin, TX) equipped with a data acquisition board (DT3003/PGL, Data Translations, Marlboro, MA) and custom Matlab software (The MathWorks Inc., Natick, MA). Stimulation and recording were synchronized through custom software such that electrical stimulation coincided with the initiation of a 20 ms recording period.

Mechanical nociceptive sensitivity tests: All animal procedures were performed in strict accordance with the Animal Studies Committee and the Division of Comparative Medicine at Washington University School of Medicine (protocol #20180103). In devices constructed for multi-day freely moving animal experiments, connections to a bioresorbable microfluidic evaporative cooler and temperature sensor mounted to the sciatic nerve (FIG. 24, panel B) route subcutaneously along the spine to a headcap (FIG. 24, panel C). Silicone microfluidic tubing and coiled electrical interconnects provide stretchability to accommodate natural motions of the spine (FIGS. 24, panels D-F). Interconnects that extend along the spine are non-bioresorbable but can be extracted without surgery after dissolution of the nerve cooler. An integrated connecter mounted inside a titanium headcap enables reversible fluidic and electronic connection to an awake animal (FIGS. 24, panels G-I).

Incising the lateral surface of the thigh and sectioning through the biceps femoris muscle exposed the sciatic nerve and its three terminal branches: the sural, common peroneal and tibial nerves. Axotomy and tight ligation of the tibial and common peroneal nerves with 6.0 silk sutures while leaving the sural nerve intact formed the spared nerve injury. Sectioning the peroneal and tibial nerves distal to the ligation and removing 2-4 mm of the distal nerve stump prevented nerve regrowth. Care was taken to avoid contact with or stretching the intact sural nerve. Muscle and skin were closed in two layers.

A light shielded enclosure with a wire mesh floor served as the environment for measuring mechanical sensitivity. Placing the animals in the test environment 30 min before measurements began acclimated the animals. Mechanically stimulating the lateral portion of the planter surface (innervated by the sural branch) of the paw with a von Frey anesthesiometer (IITC Life Science, 800 g, rigid tips) on the SNI and contralateral side while cooling the nerve yielded force values at 36, 20, and 10° C. The increase in mechanical sensitivity is represented by a decrease in the force required to elicit a sensation. Two animals received only the SNI and served as controls. Three animals received both the SNI and the cuff on the same side. Contralateral nerves served as a control and received neither the SNI nor cooling cuff. 10 measurements on the SNI and contralateral sides were taken with resting periods of 3 min in between measurements. The minimum force values for each animal were averaged and represent the mechanical sensitivity threshold for each cohort. Data was obtained three weeks after implantation (n=3). Data points and error bars represent means±S.E.M. Statistical comparisons between the SNI and cuff side and contralateral were determined by paired, two-tailed t-test (*P<0.05, ns=not significant).

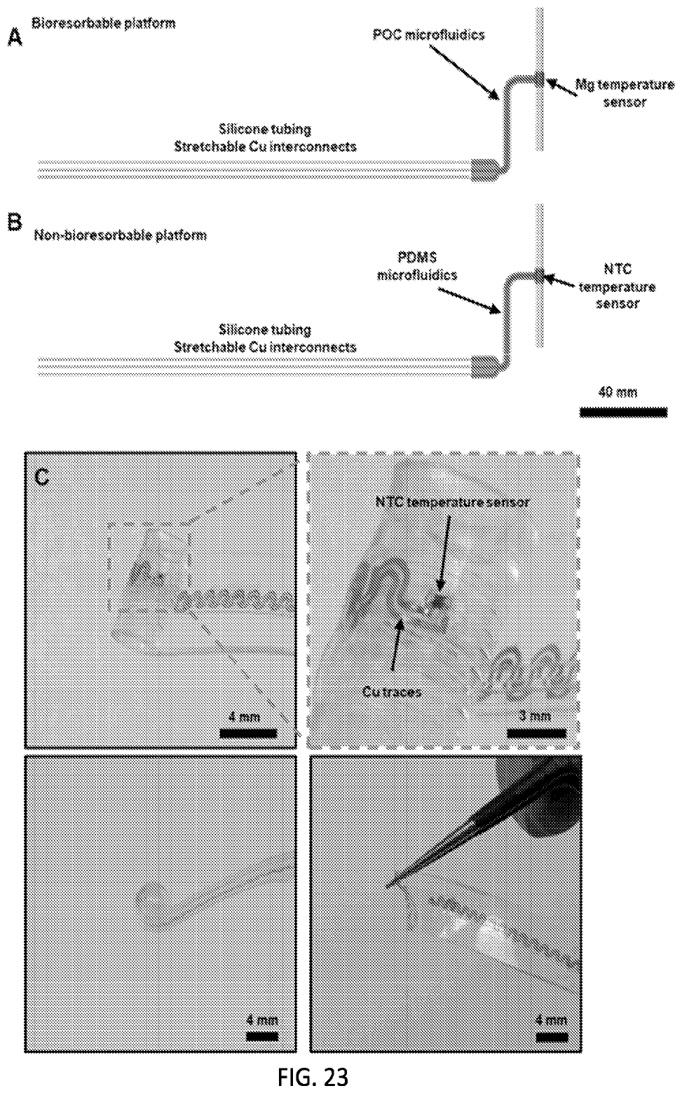

Non-bioresorbable nerve coolers consisting of silicone microfluidics and a commercial temperature sensor with comparable geometries and thermal properties yield capabilities for cooling and measuring nerve temperature for mechanical nociceptive sensitivity tests (FIG. 23). Data from commercial temperature sensors confirmed that cooling effects remained consistent for the duration of these trials.

Assessment of biocompatibility: Rat sciatic nerve tissue and transient nerve cuffs were explanted and fixed in 10% neutral buffered formalin for 48 hrs. Nerve tissue was bisected at the transverse midline of the cuff, wrapping the sciatic nerve. The sciatic nerve was then processed using a Tissue TEK VIP6 processor. After processing, tissues were paraffin embedded with transverse cut side down into the FFPE block. Sections at 4 μm thick were cut with a Thermo Scientific HM355S Microtome at the site of the nerve/cuff interface and stained with H&E and toluidine blue, respectively. All time points were performed in triplicate.

Comparison of methods for delivery of cooling power in vivo: The delivery of pre-cooled fluid through looped metal or silicone tube interfaces to ex vivo nerve preparations, and in a few cases in awake, freely moving animals shows some promise for cooling induced nerve conduction block. Here, reductions in temperature localized at the anatomical site of interest follow from actively controlled mass transfer of liquid coolant to this region through systems with thermal insulation to mitigate parasitic thermal losses along the fluidic delivery pathway. As an alternative, thermoelectric modules can apply cooling power directly at a desired location, as demonstrated with tissues of the cortex and sub-cortical structures. Rigid and bulky form factors, high power requirements, and the production of waste heat preclude use in peripheral nerves of freely moving animals. As a result, these and other mechanisms cannot provide precise cooling to small volumes of neural tissue in formats compatible with long term implantation. In addition, all previously reported technologies require surgical extraction after a period of need.

Key advances demonstrated here are in the use of a liquid to gas phase transition as a cooling mechanism and in soft microfluidic structures as a bioresorbable delivery platform. Here, elastomeric microfluidic channels route microliter volumes of a bioinert coolant to peripheral nerves where flow of a dry gas triggers local and fully contained evaporation of the coolant. The enthalpy, or latent heat, of evaporation generates the cooling effect. This approach presents significant advantages for rapidly creating large-amplitude, localized cooling effects with relaxed fluid delivery requirements as compared to traditional techniques that rely on forced liquid convection. Specifically, the cooling power associated with this scheme can exceed that of forced convection by one to two orders of magnitude. In addition, the use of a compressible working fluid lowers the operating pressures and the occurrence of transient pressure spikes, by comparison to systems that use forced convection. These unusual operating parameters enable the use of thin, soft bioresorbable materials for microfluidic delivery structures that can also integrate with flexible electronic sensors for measuring nerve temperature. As a consequence, this type of evaporative cooling, previously established in the context of microelectronic thermal management and lab-on-chip systems, can now be exploited as implantable, bioresorbable interfaces to small, soft tissue structures such as peripheral nerves.

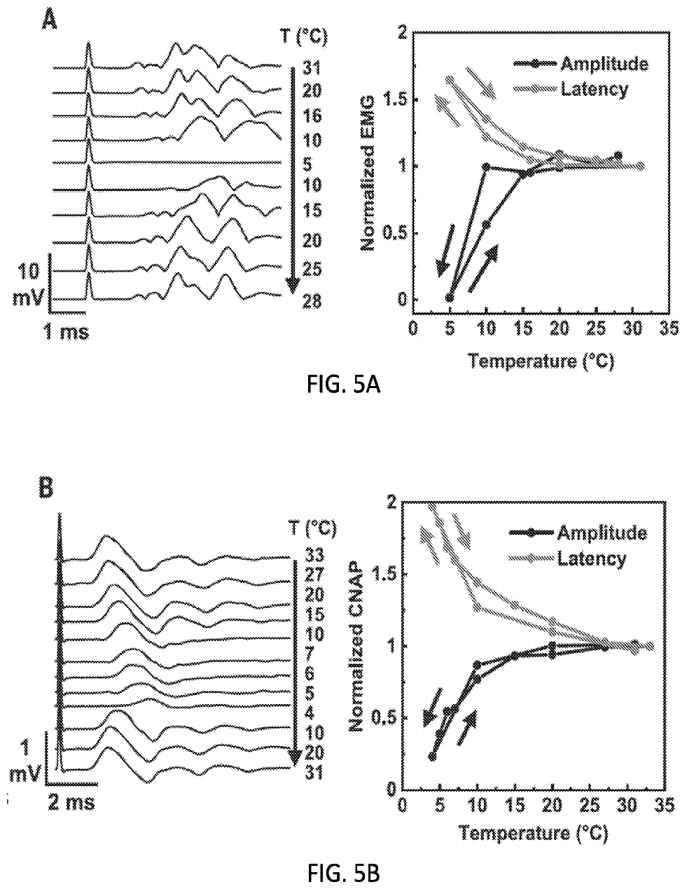

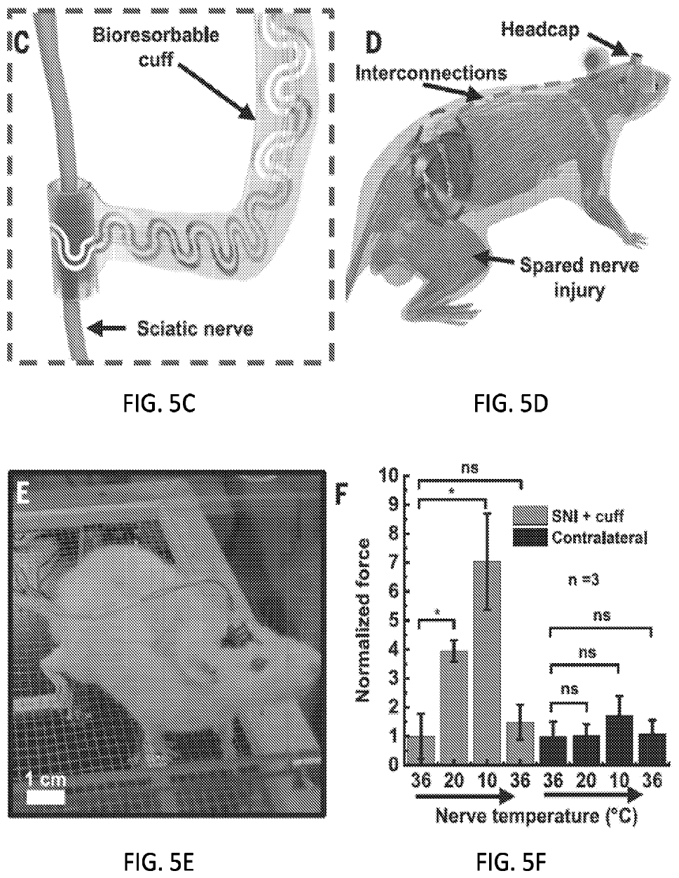

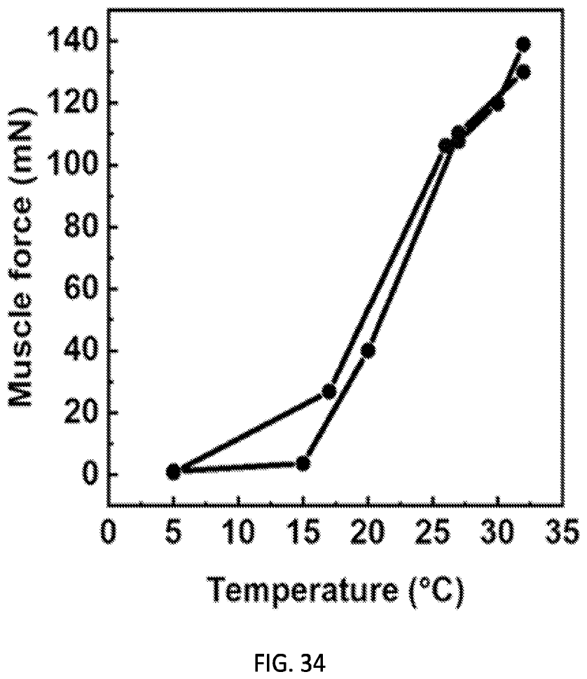

Acute nerve block studies: Electromyography (EMG) of the tibialis anterior muscle indicates elimination of neuromuscular activity during cooling from 31° C. to 5° C. over a period of 8 min (FIG. 5A). The amplitude of the EMG signal nerve distal to the cooling cuff provides a measure of compound nerve action potential evoked via a single stimulation pulse (1 mA, 50 μs). Cooling from 33° C. to 4° C. over a period of 15 min prompts a decrease in signal amplitude of 77% (0.65 mV to 0.15 mV) and increase in latency of 97% (1.8 ms to 3.5 ms) (FIG. 5B). Amplitude and latency return to within 101% (0.66 mV) and 97% (1.7 ms) of their initial values, respectively, after subsequent rewarming over a period of 3 min. Measurements of evoked muscle force in the extensor digitorum longus (EDL) muscle in response to a single stimulation pulse (1 mA, 50 μs) during cooling from 32° C. to 5° C. indicate that cooling eliminates muscular activity (140 mN to 0) over a period of 8 min (FIG. 34). Subsequent rewarming to 32° C. over a period of 3 min prompts recovery of 93% of the initial muscle force (130 mN).

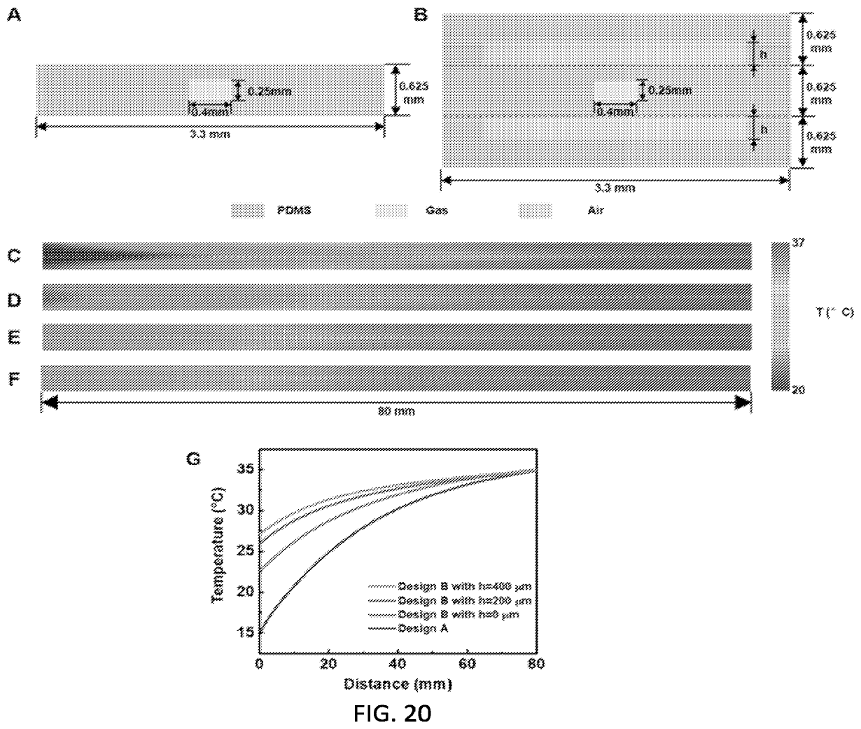

Cooling along exhaust channel: The minimum temperature at the device/tissue interface along the section of the device that contacts muscle tissue is 15° C., as revealed by simulation (FIG. 20). The minimum temperature along the spine is approximately 22° C. One option to minimize this off-target cooling is to add thermally insulating structures along the exterior of the exhaust channel. Thermal simulations demonstrate that the addition of thin air pockets on either side of this channel can minimize the temperature change at the device/tissue interface by up to 15° C. Specifically, this addition limits off-target cooling in the muscle and spine to 27° C. and 31° C., respectively. Previous studies demonstrate that human muscle maximal twitch contractions are largely unchanged when cooled to 25° C., indicating that off-target cooling of this magnitude may be well tolerated. Cooling cat spinal cords to 32° C. produces only mild changes in conduction velocity, suggesting that this level of cooling could be well tolerated.

### Results and Discussion

The technology includes a hybrid microfluidic and electronic system for cooling and simultaneously measuring the temperature of a peripheral nerve. The elastomeric nature of the micro-fluidic system and the serpentine shapes of the electrical interconnects yield soft, stretchable mechanics at the device level (FIG. 1D) with effective moduli not substantially higher than those of peripheral nerves (rat sciatic nerve elastic modulus is 0.6 MPa). These electronic and microfluidic systems terminate in a cuff structure with a diameter matched to the rat sciatic nerve (1.5 mm) to provide an intimate mechanical and thermal interface to the nerve, without the need for sutures (FIG. 1E). The curled geometry of the cuff and its elastic nature enables manual unrolling and soft clasping of the nerve. An essential defining characteristic of this system is that it is constructed entirely with water-soluble constituent materials that controllably dissolve to biocompatible end products in the biofluids that are contained in subcutaneous tissues. FIG. 1F shows devices wrapped around a silicone phantom nerve and submerged in phosphate-buffered saline (PBS) (pH 7.4) at 75° C., as an accelerated aging test. The results show that the materials largely dissolve within 20 days and that elimination of residues occurs after 50 days under these conditions.

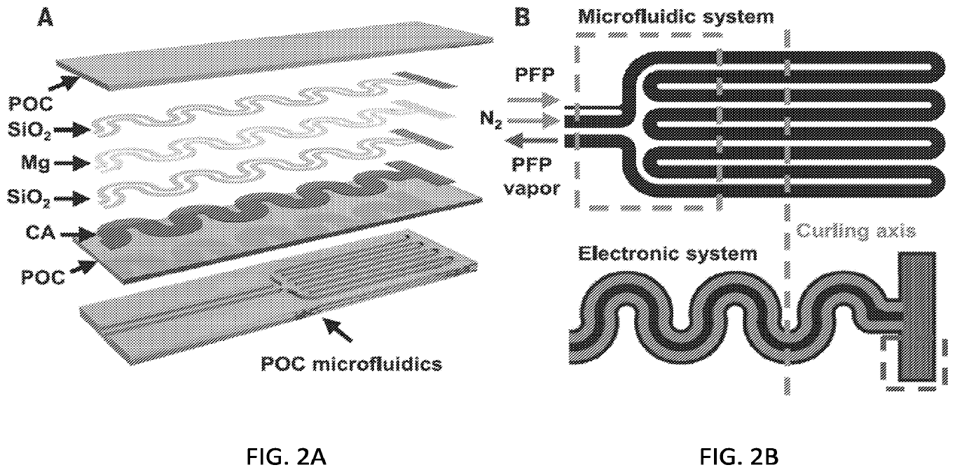

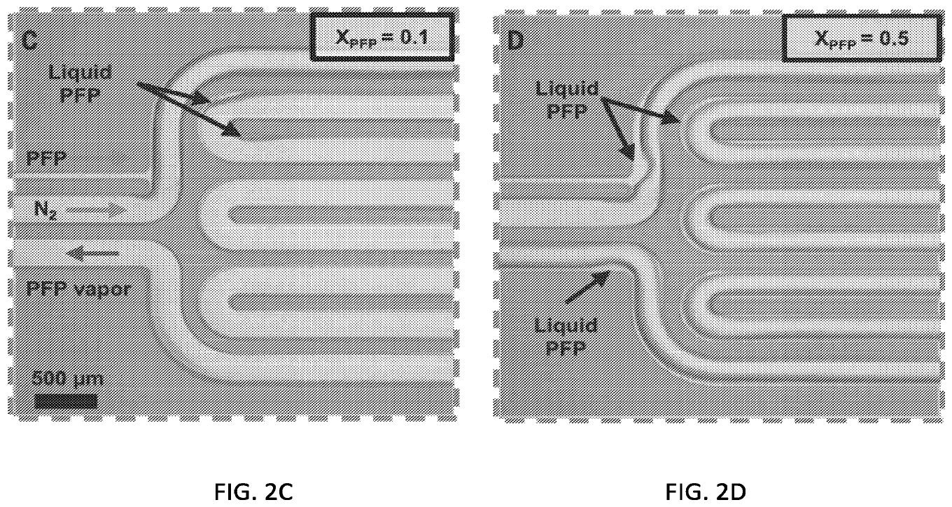

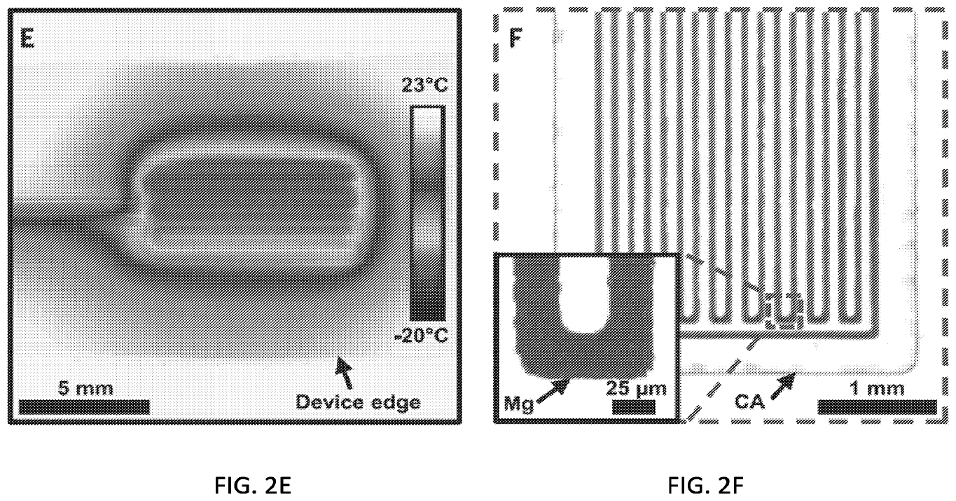

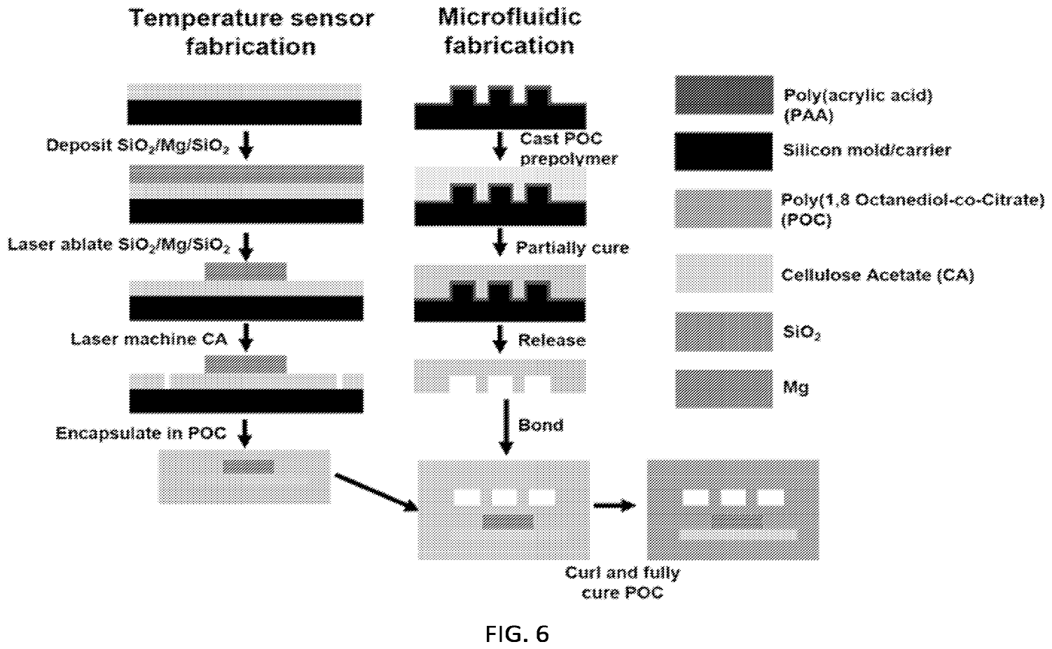

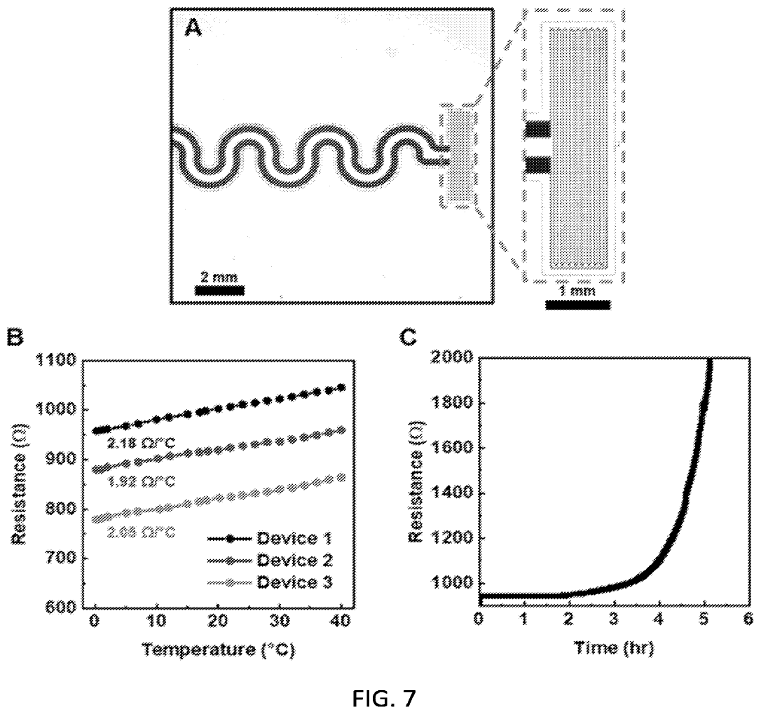

An illustration of the layers of the hybrid microfluidic-electronic device is shown in FIG. 2A. A bioresorbable elastomer, poly(octanediol citrate) (POC), forms the microfluidic system. POC exhibits an elastic modulus of 2.8 MPa, controllable rates of degradation by means of surface erosion, and a demonstrated compatibility with nerves. Details of fabrication are shown in FIG. 6 and the section of Materials and Methods. The microfluidic system includes transcutaneous colinear interconnects that deliver liquid coolant (perfluoropentane (PFP)) and dry N₂ to a serpentine evaporation chamber (FIG. 2B) in a completely sealed system that provides fluidic access at the ends. The electronic layer lies coplanar with the microfluidic system, with the temperature-sensing element at the distal end of the device. The simultaneous initiation of PFP and N₂ flows into this structure prompts evaporation of PFP at the microfluidic junction between the PFP and N₂ channels and along the serpentine chamber. PFP, which boils near room temperature (28° C. to 30° C.), is bioinert and compatible with nonfluorinated elastomers. PFP is clinically approved as a propellant in pressurized metered-dose inhalers, as an intravenous ultrasound contrast agent, and in therapeutic hypothermia. The mass flow rates of the PFP and N₂ and the geometry of the evaporation chamber govern the magnitude and localization of the cooling effect. At low PFP molar flow ratios (X_{PFP}=0.1), the PFP fully evaporates after passing through three serpentines, with marginal liquid PFP buildup at the corners of the microchannels (FIG. 2C). At high molar flow rates (X_{PFP}=0.5), PFP proceeds through annular flow and passes along the sidewalls of the microchannels (FIG. 2D). This phase change prompts the temperature of a device in a planar, uncurled configuration to drop to −20° C. within 2 min after initializing flow in ambient, room-temperature conditions (FIG. 2E). The cooled area of the device is confined predominately to the serpentine evaporation chamber, as governed by the microfluidic channel design and fluid flow rates. A serpentine magnesium trace with a width and length of 25 μm and 72 mm, respectively, provides temperature feedback through the temperature coefficient of resistance of Mg (FIG. 2F). Details for the temperature-sensing system are provided in FIG. 7 and the section of Methods and Materials.

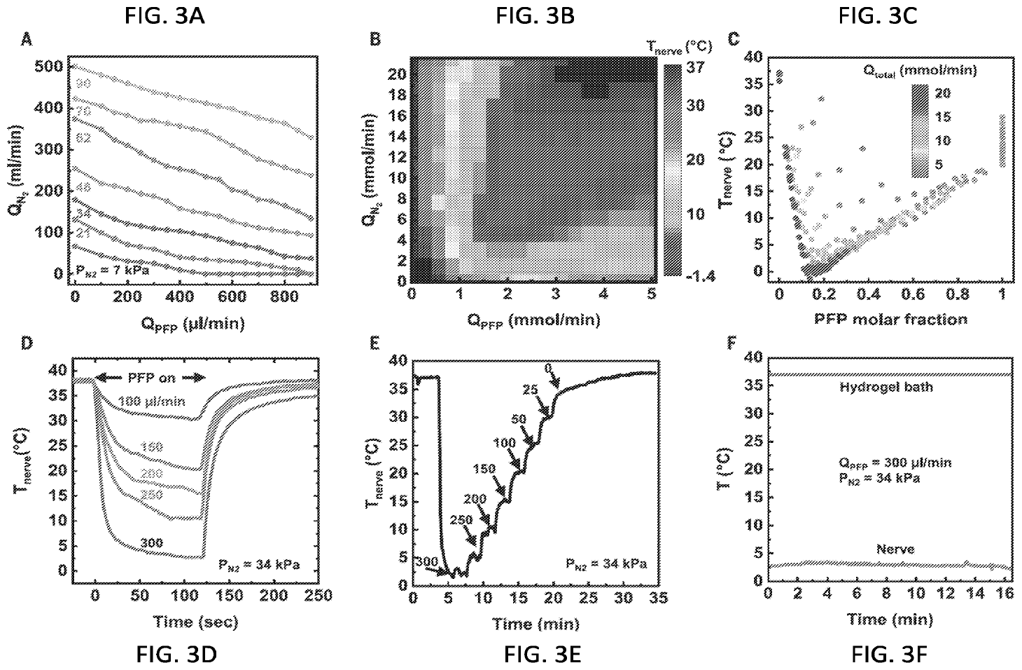

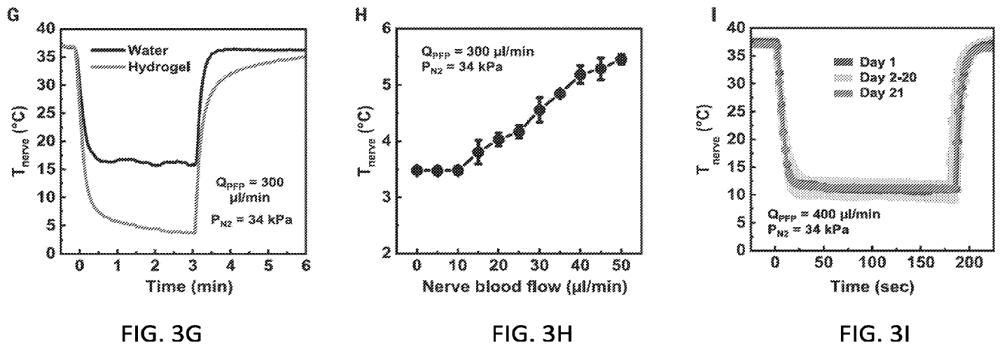

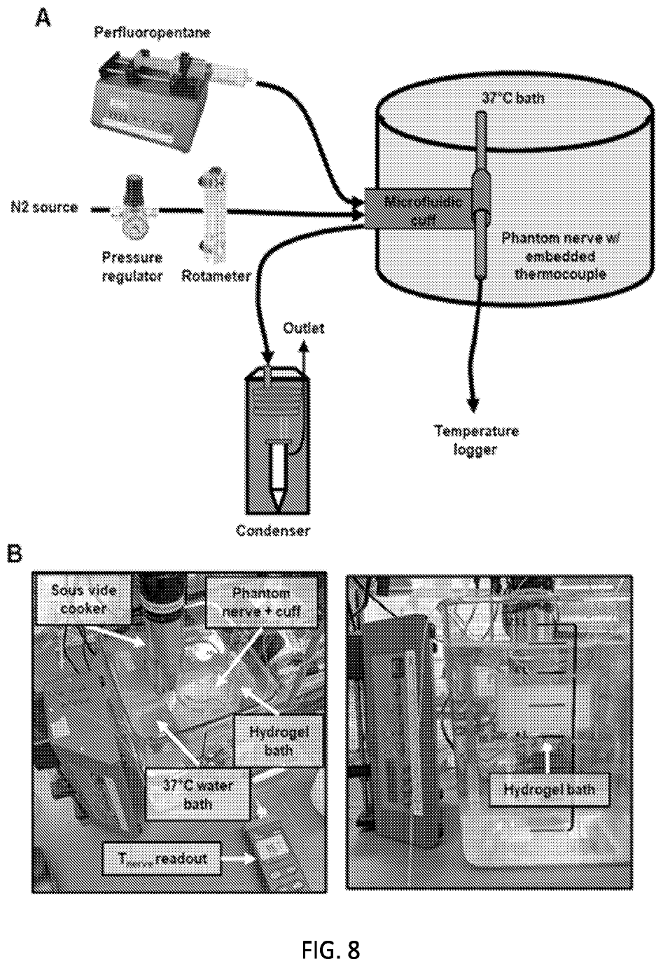

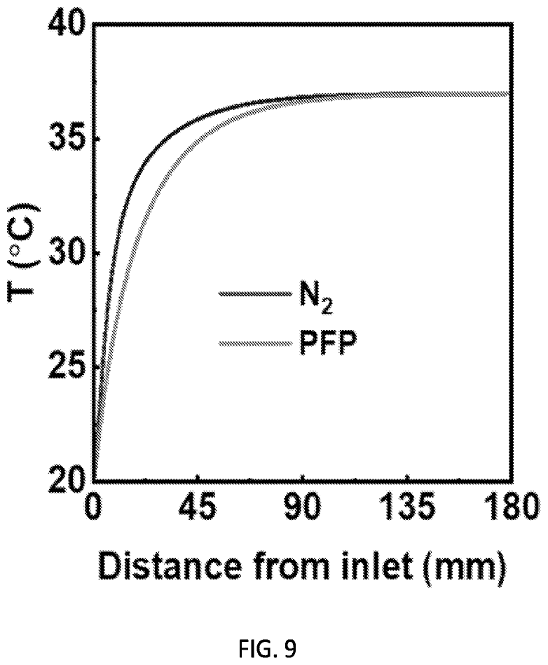

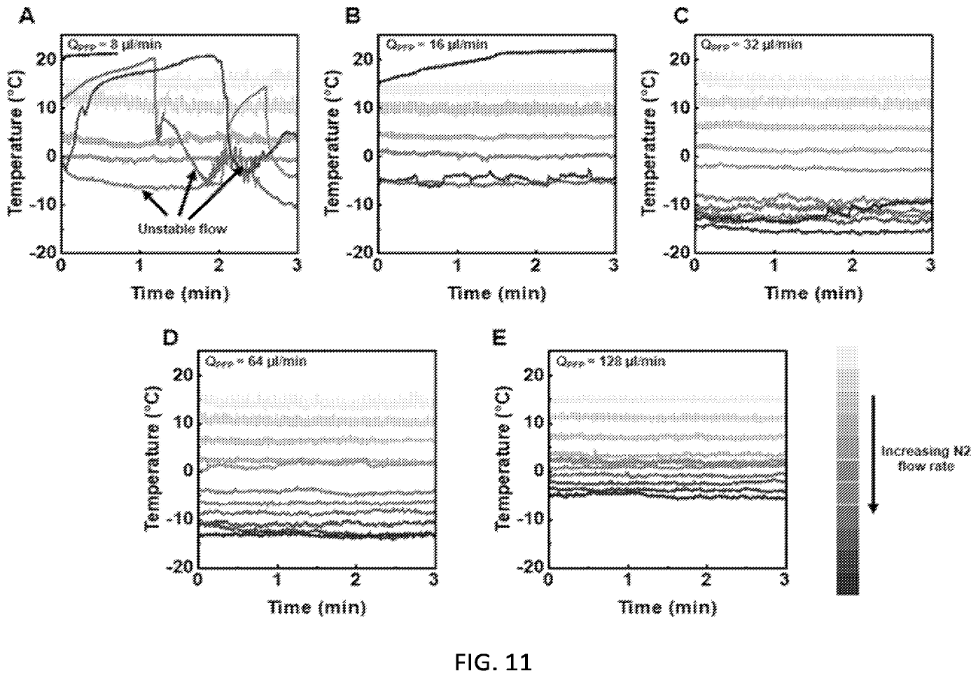

FIGS. 3A-3I summarize quantitative results of measurements of the efficacy of nerve cooling of a phantom nerve structure and an integrated thermocouple inside a 37° C. hydrogel tissue mimic (FIG. 8). An enabling property of PFP is that it can be superheated beyond its boiling point, to temperatures as high as 60° C., because of the energy barrier required for homogeneous nucleation. This feature prevents premature boiling of the PFP as it passes along the smooth interior of the microfluidic channels to the evaporation chamber. The temperatures of the PFP and N₂ flows equilibrate at 37° C. within 90 mm of entering the 37° C. environment (FIG. 9). A condenser placed distal to the cooling cuff enables recapture of 90% of the evaporated PFP (FIG. 10). The delivery of N₂ in a pressure-driven mode mitigates transient pressure spikes that can occur with multiphase microfluidic flows (FIG. 11).

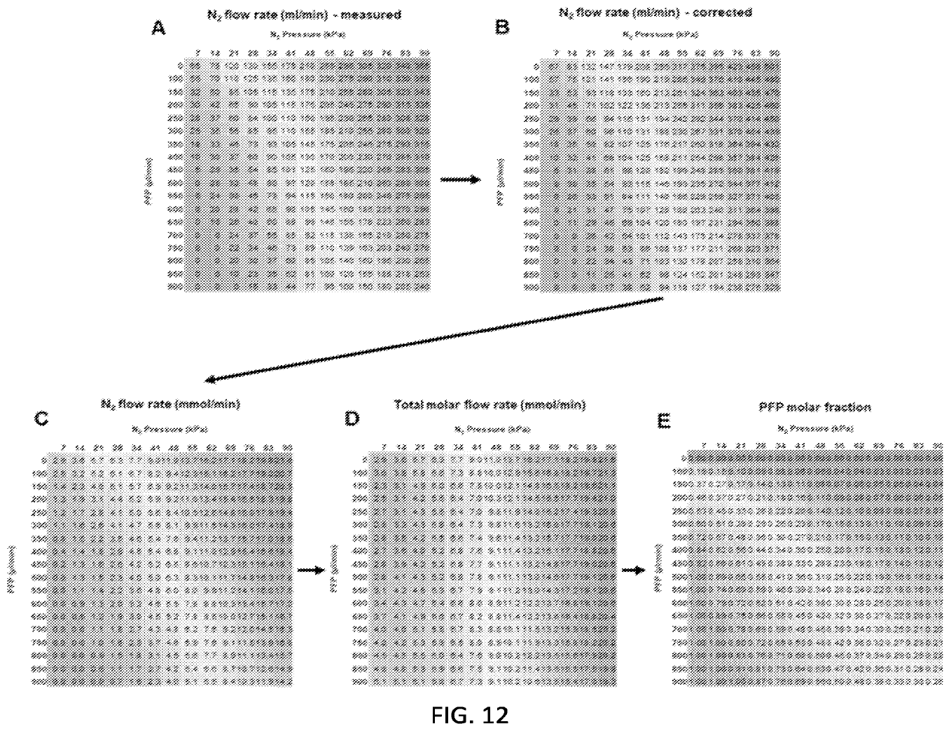

Holding the N₂ pressure constant at values ranging from 7 to 90 kPa and sweeping the PFP flow rates from 0 to 900 μl/min yields data that reveal the dependence of the N₂ flow rate on PFP flow rate (FIG. 3A). Experiments based on systematically sweeping the PFP and N₂ flow rates over the same range as in FIG. 3A reveal the effect of molar flow rates on resultant nerve temperature (FIG. 3B). The minimum nerve temperature is −1.4° C., which is achieved at a molar PFP fraction of 0.13 and a N₂ pressure of 90 kPa (FIG. 3C). Additional details regarding conversion to molar flow rates are provided in FIG. 12. FIG. 3D demonstrates the effect of PFP flow rate on nerve-temperature cooling rate. The maximum cooling rate of 3° C./s occurs with the 300 μl/min case. Arbitrary cooling and rewarming profiles, as governed by the thermal properties of the surrounding media, follow from controlled increments or decrements of the PFP flow rate (FIG. 3E). Experiments indicate sustained and consistent nerve cooling to 3.0° C. for a 15-min interval in FIG. 3F (minimum temperature (Tₘᵢₙ)=2.1° C.; maximum temperature (Tₘₐₓ)=3.5° C.).

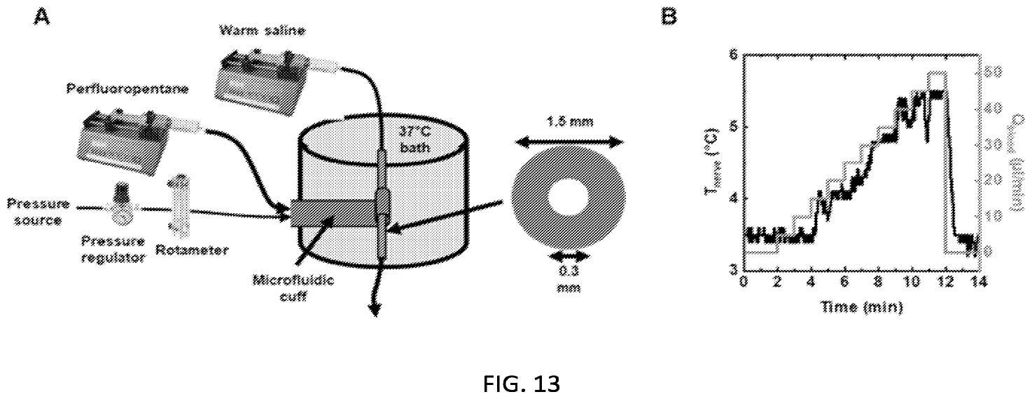

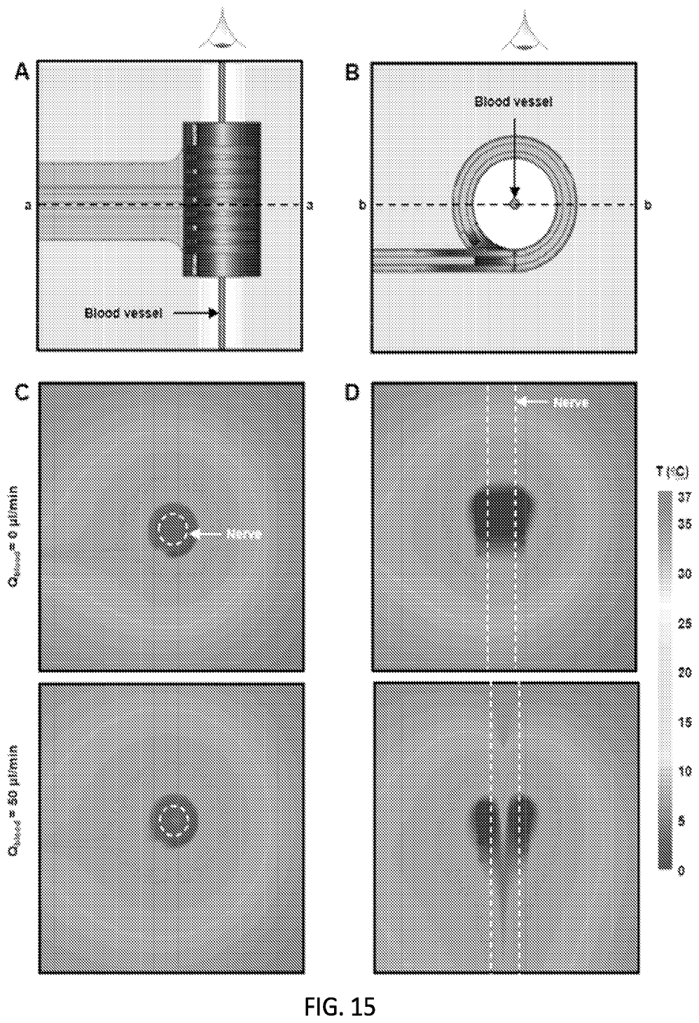

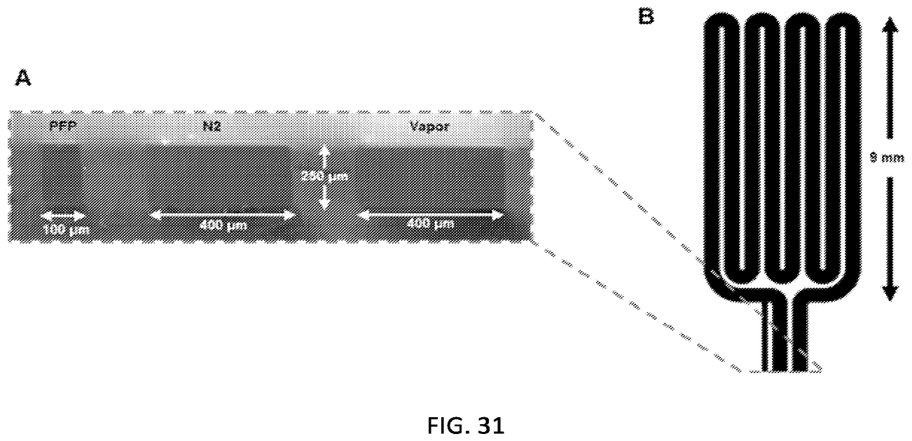

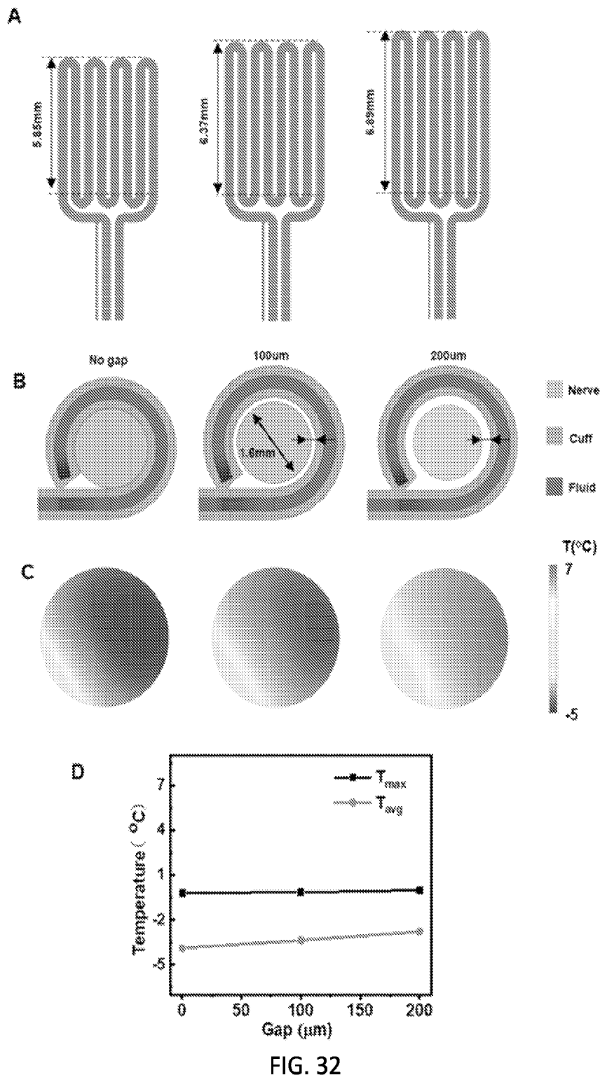

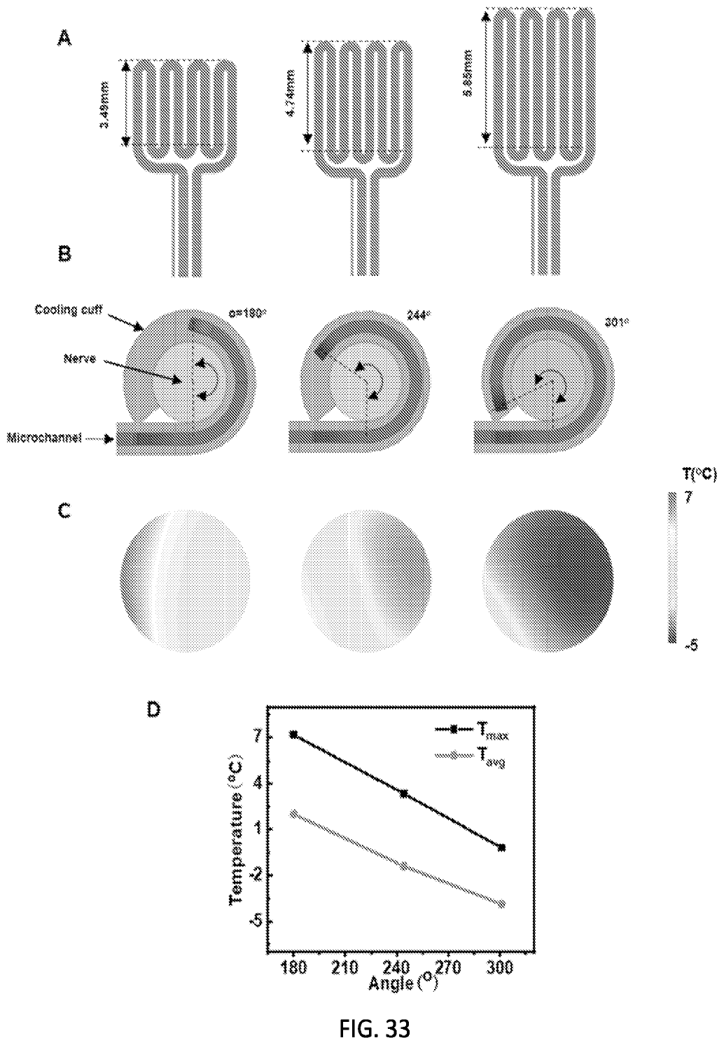

Thermal transfer in nerves occurs mainly through conduction, though subcutaneous biofluid and nerve blood flow contribute to convective thermal transfer. Comparisons of measurements in a conduction-only environment (hydrogel, 37° C.) to those that include convective effects (water, 37° C.) highlight these effects (FIG. 3G). The perfusion of blood through the targeted nerve presents an additional source of heat flux. For a nerve blood flow of 50 μl/min (FIG. 3H), the temperature of the nerve increases by 2.0° C. (from 3.5° C. to 5.5° C.). Experimental details are provided in FIG. 13. A thermofluidic model (FIG. 14) predicts the resultant changes in nerve temperature with and without nerve blood flow and shows similar results to experimental results (FIG. 15). Details for the thermofluidic model are provided in the section of Methods and Materials. Data captured for daily cooling for 180 s over 21 consecutive days indicate stable operation in 37° C. PBS, with a standard deviation in cooling temperature of 0.6° C. (FIG. 31).

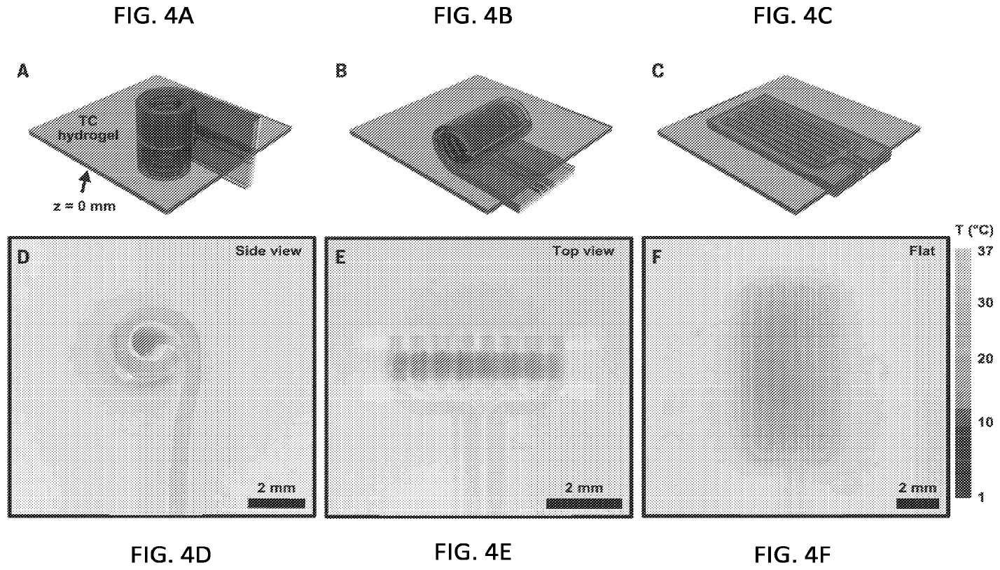

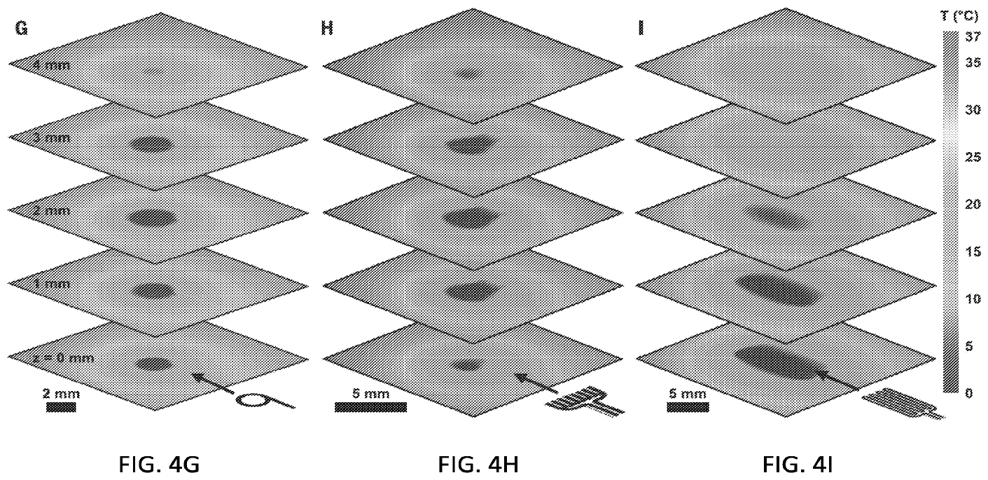

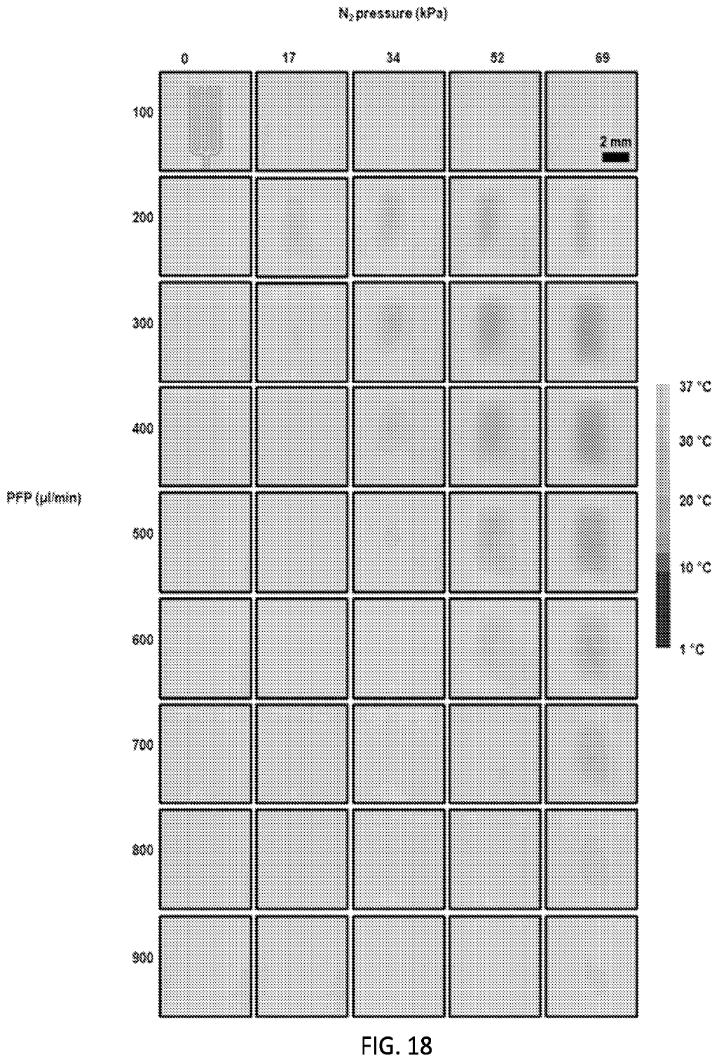

Localization of cooling effect: Localization of the cooling effect to a pre-defined site without the need for insulating layers represents a key capability of the evaporative microfluidic cooling approach introduced here. Nerve coolers embedded in a thermochromic tissue mimic in three different configurations serve as models for experimental study of temperature gradients in radial (FIG. 4A) and longitudinal (FIG. 4B) views along the nerve and across the surface of an uncurled, planar device (FIG. 4C). FIG. 16 illustrates the experimental setup for quantifying the 10° C. thermocline in similar views of curled devices and a top-down view of a flat device. The 10° C. thermocline is contained within the cuff (FIG. 4D) except for a region that extends downward ˜500 mm from the exterior of the cuff and does not extend past the edge of the device longitudinally along the nerve (FIG. 4E). FIG. 4F demonstrates that this thermocline extends over nearly the entire serpentine evaporation chamber in the flat device. Images of flow rate sweep experiments for the cases shown in FIGS. 4E-4F are shown in FIGS. 17-18, respectively. Thermal three-dimensional finite element analysis confirms that the cooling effect is largely confined radially (FIG. 4G) and longitudinally (FIG. 4H) within the extent of the cooling cuff and above a flat cooler (FIG. 41). The temperature of the vapor remains confined inside the microfluidic channel, as indicated by the cold region that extends radially down and to the right in the z=0 mm plane for FIG. 4G.

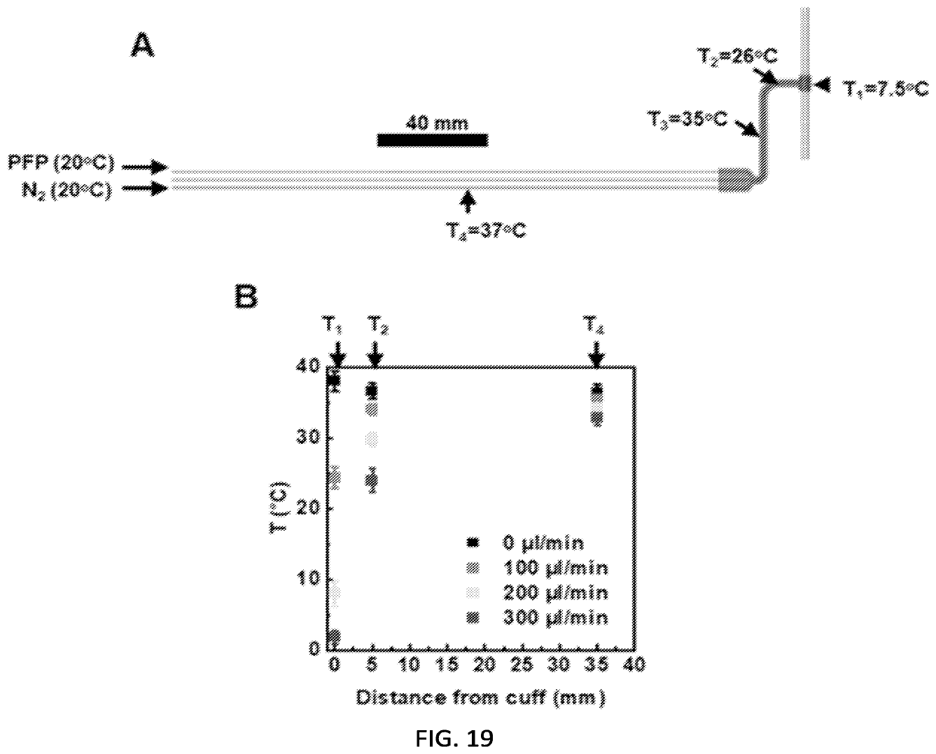

The low heat capacity of the PFP and N₂ exhaust gas mixture yields minimal cooling along the exterior of the outlet channel, as supported by experiments and simulations (FIGS. 19-20).

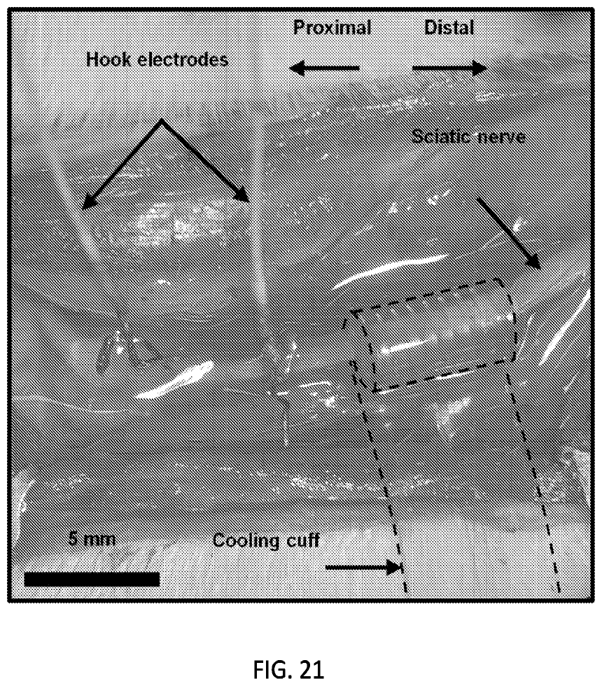

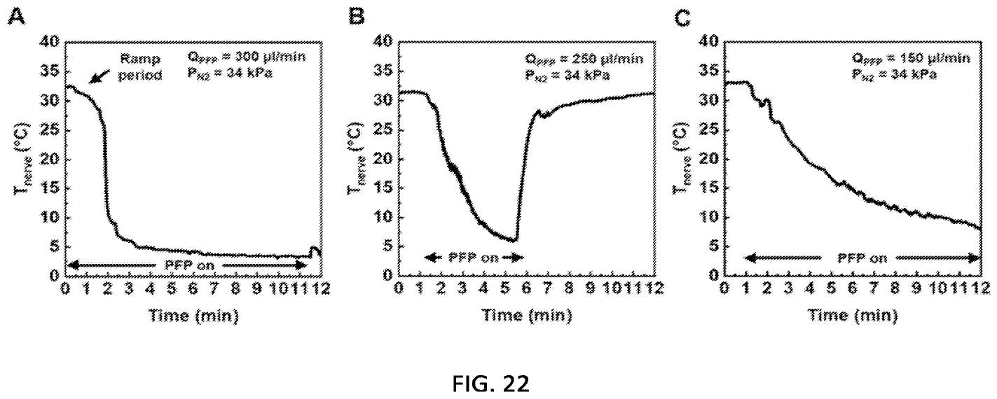

Acute nerve block studies: Acute animal trials demonstrate the capability of evaporative microfluidic coolers to reversibly eliminate evoked nerve signals. The soft, curled structure defines an intimate thermal and mechanical interface to the rat sciatic nerve without sutures and without mechanically induced damage (FIG. 21). Electromyography (EMG) of the tibialis anterior muscle indicates a 92% reduction in EMG magnitude and 64% increase in signal latency of neuromuscular activity during cooling from 31° C. to 5° C. over a period of 8 min (FIG. 5A). The amplitude and latency return to 108 and 100% of their initial values, respectively, after rewarming over a period of 3 min. Electrical recording from the sciatic nerve distal to the cooling cuff provides a measure of compound nerve action potential (CNAP) evoked through a single stimulation pulse. Cooling from 33° C. to 4° C. over a period of 15 min prompts a decrease in signal amplitude of 77% and increase in latency of 97% (FIG. 5B). Amplitude and latency return to within 101 and 97% of their initial values, respectively, after subsequent rewarming over a period of 3 min. Examples of in vivo cooling profiles are provided in FIG. 22.

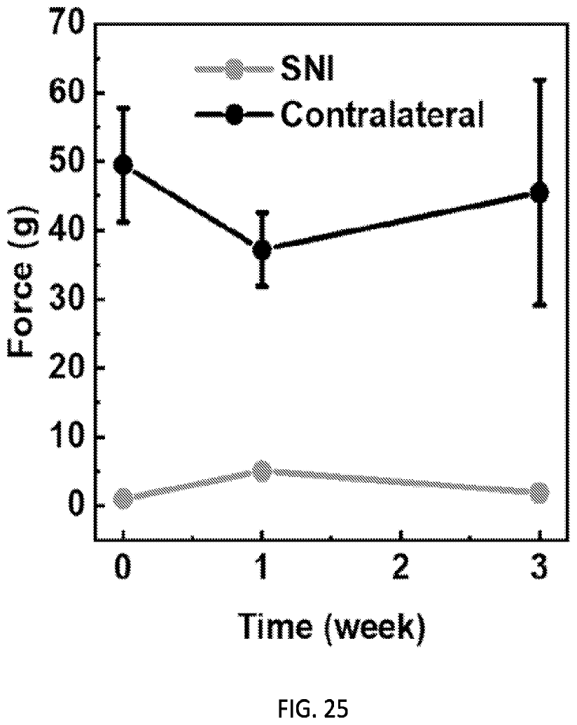

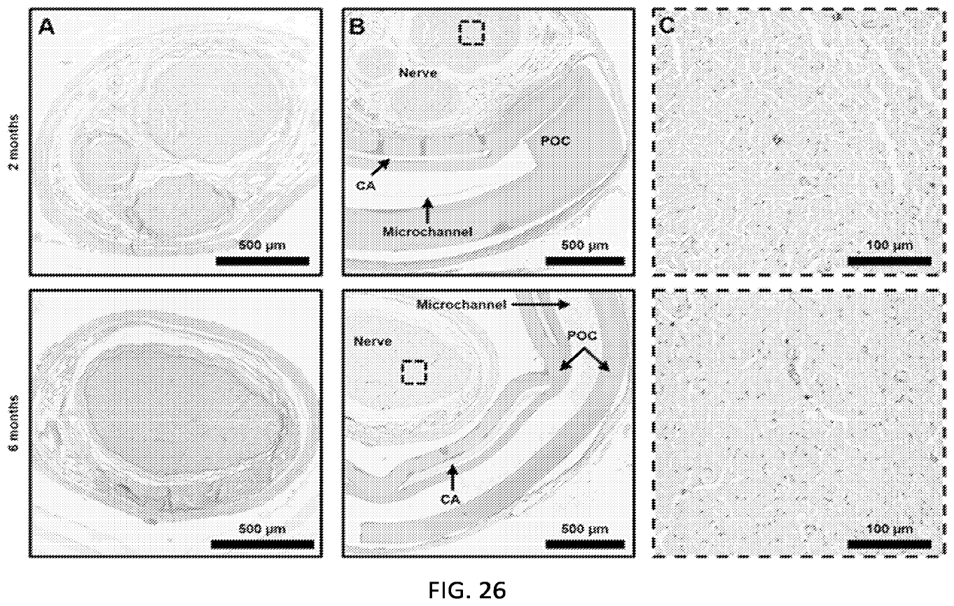

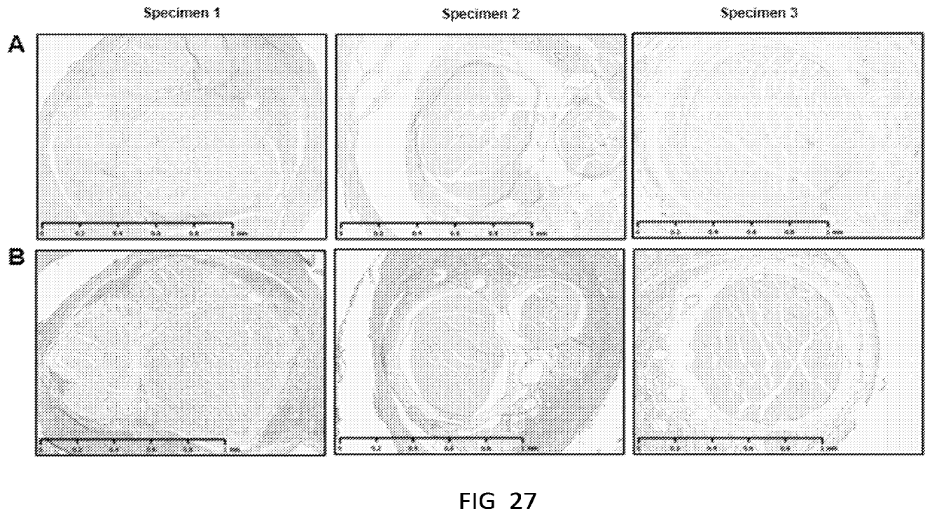

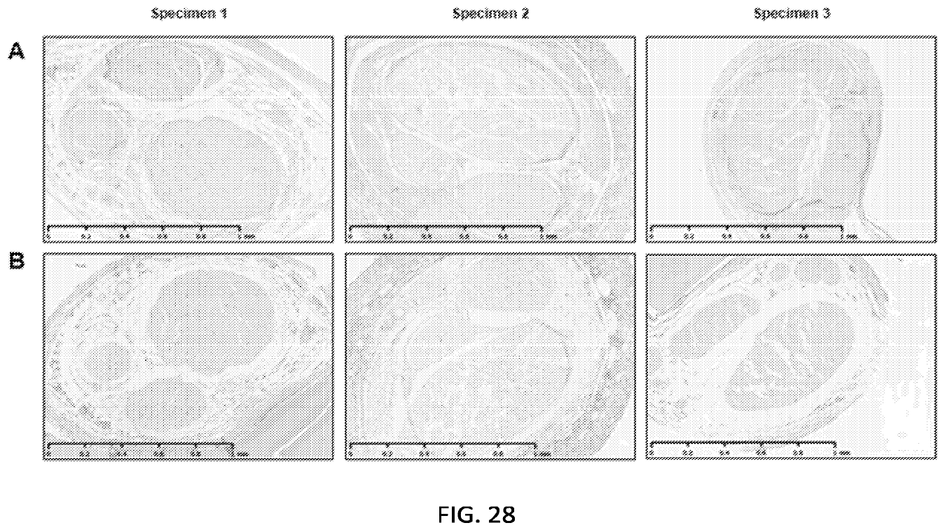

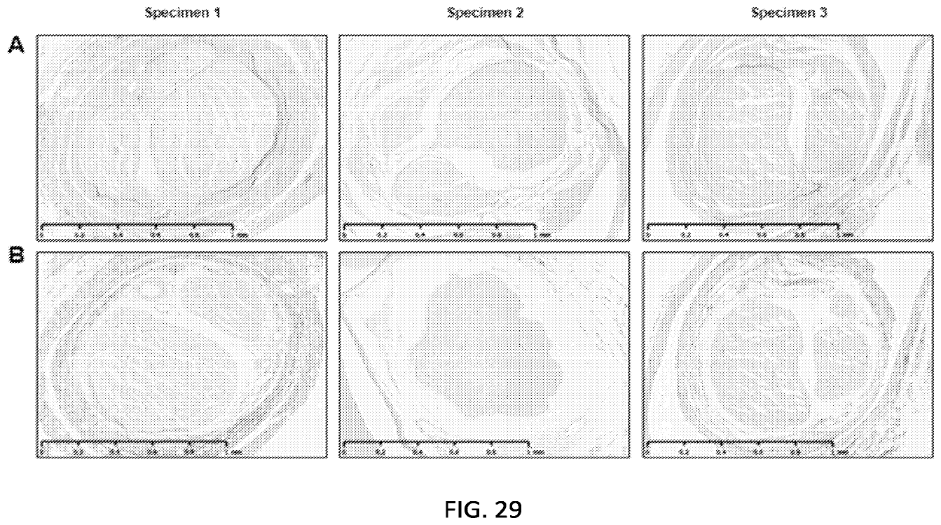

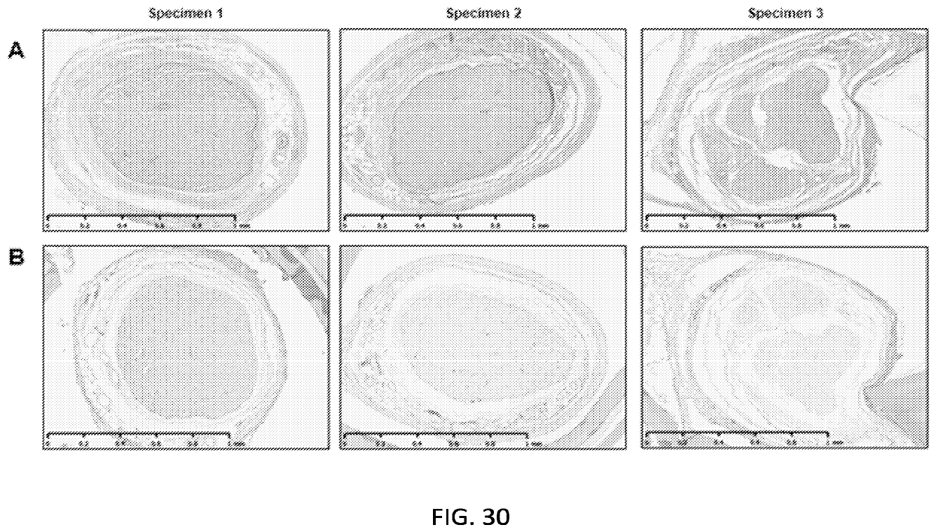

Cooling-induced analgesia in freely moving animals: Experiments based on cooling of sciatic nerves in a rat model of neuropathic pain associated with spared nerve injury (SNI) elucidate capabilities for microfluidic evaporative coolers to block pain in freely moving animals. In devices constructed for multiday experiments, transcutaneous connections to a bioresorbable microfluidic evaporative cooler and temperature sensor mounted to the sciatic nerve (FIG. 5C) route subcutaneously along the spine to a headcap (FIG. 5D). Device details are provided in FIGS. 23-24. An integrated connecter mounted inside a titanium headcap enables reversible fluidic and electronic connection to an awake, freely moving animal (FIG. 5E). Mechanical nociceptive sensitivity tests in two control animals (SNI only) over 3 weeks after the SNI show an expected increase in the mechanical sensitivity threshold of the SNI side as compared with that of the contralateral side, which persists for more than 3 weeks (FIG. 25). Studies using three additional animals that received both the SNI and the cooling cuff show that cooling of the SNI-treated nerve from 37° C. to 10° C. after 3 weeks of implantation leads to a sevenfold increase in the mechanical sensitivity threshold (1.6 g to 11.5 g), consistent with a significant cooling-induced analgesic effect (FIG. 5F). Changes in the mechanical sensitivity threshold in the contralateral side during cooling of the SNI-treated nerve are statistically insignificant. Histologic analyses after 1, 2, 3, and 6 months indicate a close proximity of the cuff to the nerve and provide evidence of biocompatibility and bioresorption, as shown in FIGS. 27-30 and Table 2. Previous in vivo studies of POC, Mg, SiO₂, and cellulose acetate provide additional strong evidence of biocompatibility and associated bioresorption processes.

TABLE 2
Quantitative histologic analyses of sciatic nerves with cuffs for 1, 2, 3, and 6 months.
Cuffs were well tolerated for up to 6 months with minor, if any, evidence of axon damage or
acute or chronic inflammation. Each time point was performed in triplicate. Values are denoted
as the % of total affected nerve surface area.

| | Axon | Neutro- | Lympho- | Eosino- | | Plasma | |
|---|---|---|---|---|---|---|---|
| Specimen | damage | phils | cyes | phils | Ischemia | cells | Notes |
| 1MoN1 | <1% | <1% | 6.36% | <1% | None | 0 | Mild Perineural |
| | | | | | | | lymphocytes |
| 1MoN2 | <1º 6 | 0 | 0 | 0 | None | 0 | |
| 1MoN3 | <1% | 0 | 0 | 0 | None | 0 | |
| 2MoN1 | <1% | 0 | 0 | 0 | None | 0 | |
| 2MoN2 | <1% | 0 | 0 | <1% | None | 5% | Mild Perineural |
| | | | | | | | plasma cells |
| 2MoN3 | <1% | 0 | 0 | 0 | None | 0% | |
| 3MoN1 | <1% | <1% | 0 | <1% | None | <1% | Perineural plasma |
| | | | | | | | cells. Numerous |
| | | | | | | | neutrophils. however, |
| | | | | | | | appear in_vessels and |
| | | | | | | | not in contact with |
| | | | | | | | nerve |
| 3MoN2 | <1% | 0 | <1% | 0 | None | 0 | Single intraneural |
| | | | | | | | lymph |
| 3MoN3 | <1% | 0 | | 0 | None | 0 | Rare multinucleated |
| | | | | | | | giant cells (2 total) |
| 6MoN1 | <1% | 0 | <1% | 0 | None | <1% | |
| 6MoN2 | <1% | 0 | 0 | <1º b | None | 0 | Rare multinucleated |
| | | | | | | | giant cell and |
| | | | | | | | occasional penneural |
| | | | | | | | eosinophils |
| 6MoN3 | <1° C. | 0 | 0 | 0 | None | 0 | |

### CONCLUSION

The results presented here demonstrate that spatiotemporally precise cooling enabled by soft, bioresorbable evaporative microfluidics provides for reversible elimination of local peripheral nerve activity. Experiments indicate capabilities in on-demand analgesia for the management of neuropathic pain in freely moving animal models. These concepts, materials, and device designs establish the engineering foundations for a class of implantable cooling systems capable of targeted neural blocking with relevance across a range of clinical applications, including targeted, on-demand, nonopioid pain management.

Opportunities for improvements lie in 1) the development of approaches to produce a differential block of sensory activity, 2) techniques to establish precisely defined time-dynamic nerve cooling profiles that yield safe and beneficial outcomes, and 3) strategies to miniaturize the fluid management system, as discussed in detail in the following.

First, clinical pain management requires that motor function remains unaffected while blocking transmission in small diameter, unmyelinated nerve fibers that are primarily responsible for carrying signals related to pain. Some studies suggest that small diameter, slower-conducting sensory fibers exhibit higher temperature sensitivity than large diameter motor fibers. This difference could potentially form the basis for a scheme to selectively block sensory fibers through precise temperature control. The other option is simply to target nerves that contain only sensory fibers such as those that are traditionally targets of neurolysis or neurectomy for chronic pain or cancer pain management (e.g., sacral nerve, saphenous nerve, or celiac plexus).

Second, analgesic nerve cooling requires precise thermal and temporal control to mitigate the possibility of freezing and non-freezing tissue damage. Reduction of nerve temperature below freezing prompts ice formation that results in biochemical, anatomical, and physiological nerve pathologies. Though freezing eliminates nerve activity and is used clinically as a method of pain management, it results in a nerve block that persists for weeks to months before axonal regrowth. Cooling to temperatures above freezing can also induce nerve damage, primarily from ischemia. Intermittent cooling increases pathological features of nerve damage as compared to identical durations of continuous cooling and is primarily caused by ischemia-reperfusion injuries. These considerations necessitate additional studies to establish the analgesic window of nerve cooling as a function of cooling temperature, duration, and intermittency.

Third, the development of small, portable nerve cooling systems represents an important direction for clinical translation into settings outside of hospitals or similar care facilities. Miniaturization and integration of the pumps, reservoirs, and condensers could close the distance between the fluid handling systems and the sites of implantation to enable wearable or fully implantable units. Micropumps that exploit piezoelectric or electromagnetic actuation of deformable membranes can deliver liquids and gases at rates and pressures suitable for nerve cooling as demonstrated in this work (Q_{PFP}: 300 μl/min, Q_{N2}: 200 ml/min, P_{system}: 34 kPa). Another option for gas delivery is to use modular canisters of compressed gas. Integration of these pumps into wearable formats would enable a hybrid wearable/implantable system where skin mounted pumps deliver fluid to and from implanted nerve coolers via percutaneous leads. Peripheral nerve stimulation systems that include wearable modules and percutaneous leads for FDA approved treatment of chronic pain for up to 60 days could represent a relevant model. Development of coolant regeneration schemes that condense and recapture working fluids would serve to further miniaturize such a wearable system or potentially enable fully implantable, closed-loop nerve cooling. The cumulative volume of these fluid handling components is comparable to that of intrathecal pumps (20-40 cm³) used to deliver drugs to the spinal cord for the treatment of chronic pain.

Even in the form described here, the technology has important potential clinical utility for a range of use cases including the treatment of acute pain in hospital settings. Such engineering approaches to targeted, on-demand pain relief could bypass the need for opioids or other pharmaceutical treatments. In a broader sense, the results establish the engineering foundations for classes of bioresorbable implants capable of precision cooling-based mechanisms for neuromodulation.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. J. M. Hah, B. T. Bateman, J. Ratliff, C. Curtin, E. Sun, Chronic Opioid Use After Surgery: Implications for Perioperative Management in the Face of the Opioid Epidemic. Anesth Analg. 125, 1733-1740 (2017).
[2]. K. Yu, X. Niu, B. He, Neuromodulation Management of Chronic Neuropathic Pain in the Central Nervous System. Advanced Functional Materials. 30, 1908999 (2020).
[3]. T. R. Deer, R. Naidu, N. Strand, D. Sparks, A. Abd-Elsayed, H. Kalia, J. M. Hah, P. Mehta, D. Sayed, A. Gulati, A review of the bioelectronic implications of stimulation of the peripheral nervous system for chronic pain conditions. Bioelectronic Medicine. 6, 9 (2020).
[4] R. Melzack, P. D. Wall, Pain Mechanisms: A New Theory. Science. 150, 971-979 (1965).
[5] M. J. Cousins, D. B. M. D. Carr, T. T. M. D. Horlocker, P. O. Bridenbaugh, Eds., Cousins & Bridenbaugh's Neural Blockade in Clinical Anesthesia and Pain Medicine.
[6]. S. M. Iyer, K. L. Montgomery, C. Towne, S. Y. Lee, C. Ramakrishnan, K. Deisseroth, S. L. Delp, Virally mediated optogenetic excitation and inhibition of pain in freely moving nontransgenic mice. Nature Biotechnology. 32, 274-278 (2014).
[7]. C. Rabut, S. Yoo, R. C. Hurt, Z. Jin, H. Li, H. Guo, B. Ling, M. G. Shapiro, Ultrasound Technologies for Imaging and Modulating Neural Activity. Neuron. 108, 93-110 (2020).
[8]. V. B. Brooks, in Reviews of Physiology, Biochemistry and Pharmacology, Volume 95 (Springer, 1983), pp. 1-109.
[9]. E. H. Lothet, K. M. Shaw, H. Lu, J. Zhuo, Y. T. Wang, S. Gu, D. B. Stolz, E. D. Jansen, C. C. Horn, H. J. Chiel, M. W. Jenkins, Selective inhibition of small-diameter axons using infrared light. Scientific Reports. 7, 3275 (2017).
[10]. R. Janssen, Thermal influences on nervous system function. Neuroscience & Biobehavioral Reviews. 16, 399-413 (1992).
[11]. S. B. Rutkove, Effects of temperature on neuromuscular electrophysiology. Muscle & Nerve. 24, 867-882 (2001).
[12]. P. Borgdorff, P. G. A. Versteeg, An implantable nerve cooler for the exercising dog. Europ. J. Appl. Physiol. 53, 175-179 (1984).
[13]. Z. Zhang, T. D. Lyon, B. T. Kadow, B. Shen, J. Wang, A. Lee, A. Kang, J. R. Roppolo, W. C. de Groat, C. Tai, Conduction block of mammalian myelinated nerve by local cooling to 15-30° C. after a brief heating. Journal of Neurophysiology. 115, 1436-1445 (2016).
[14]. T. Morgan, Y. Zhang, N. Pace, H. Cai, B. Shen, J. Wang, J. R. Roppolo, W. C. de Groat, C. Tai, Thermal block of mammalian unmyelinated C fibers by local cooling to 15-25° C. after a brief heating at 45° C. Journal of Neurophysiology. 123, 2173-2179 (2020).
[15]. D. M. Ackermann, E. L. Foldes, N. Bhadra, K. L. Kilgore, Nerve Conduction Block Using Combined Thermoelectric Cooling and High Frequency Electrical Stimulation. J Neurosci Methods. 193, 72-76 (2010).
[16]. J. Jia, M. Pollock, Cold nerve injury is enhanced by intermittent cooling. Muscle & Nerve. 22, 1644-1652 (1999).
[17]. J. Jia, M. Pollock, J. Jia, Cold injury to nerves is not due to ischaemia alone. Brain. 121, 989-1001 (1998).
[18]. J. Shin, Y. Yan, W. Bai, Y. Xue, P. Gamble, L. Tian, I. Kandela, C. R. Haney, W. Spees, Y. Lee, M. Choi, J. Ko, H. Ryu, J.-K. Chang, M. Pezhouh, S.-K. Kang, S. M. Won, K. J. Yu, J. Zhao, Y. K. Lee, M. R. MacEwan, S.-K. Song, Y. Huang, W. Z. Ray, J. A. Rogers, Bioresorbable pressure sensors protected with thermally grown silicon dioxide for the monitoring of chronic diseases and healing processes. Nat Biomed Eng. 3, 37-46 (2019).
[19]. J. Koo, M. R. MacEwan, S.-K. Kang, S. M. Won, M. Stephen, P. Gamble, Z. Xie, Y. Yan, Y.-Y. Chen, J. Shin, N. Birenbaum, S. Chung, S. B. Kim, J. Khalifeh, D. V. Harburg, K. Bean, M. Paskett, J. Kim, Z. S. Zohny, S. M. Lee, R. Zhang, K. Luo, B. Ji, A. Banks, H. M. Lee, Y. Huang, W. Z. Ray, J. A. Rogers, Wireless bioresorbable electronic system enables sustained nonpharmacological neuroregenerative therapy. Nature Medicine. 24, 1830-1836 (2018).
[20]. G. H. Borschel, K. F. Kia, W. M. Kuzon, R. G. Dennis, Mechanical properties of acellular peripheral nerve. Journal of Surgical Research. 114, 133-139 (2003).
[21]. J. Yang, A. R. Webb, G. A. Ameer, Novel Citric Acid-Based Biodegradable Elastomers for Tissue Engineering. Advanced Materials. 16, 511-516 (2004).
[22]. R. T. Tran, J. Yang, G. A. Ameer, Citrate-Based Biomaterials and Their Applications in Regenerative Engineering. Annual Review of Materials Research. 45, 277-310 (2015).
[23]. R. T. Tran, W. M. Choy, H. Cao, I. Qattan, J.-C. Chiao, W. Y. Ip, K. W. K. Yeung, J. Yang, Fabrication and characterization of biomimetic multichanneled crosslinked-urethane-doped polyester tissue engineered nerve guides. Journal of Biomedical Materials Research Part A. 102, 2793-2804 (2014).
[24]. P. G. A. Rogueda, HPFP, a Model Propellant for pMDIs. Drug Development and Industrial Pharmacy. 29, 39-49 (2003).
[25]. P. Grayburn, Perflenapent emulsion (Echogen®): A new long-acting phase-shift agent for contrast echocardiography. Clinical Cardiology. 20, 12-18 (1997).
[26]. M. Castren, P. Nordberg, L. Svensson, F. Taccone, J.-L. Vincent, D. Desruelles, F. Eichwede, P. Mols, T. Schwab, M. Vergnion, C. Storm, A. Pesenti, J. Pachl, F. Guérisse, T. Elste, M. Roessler, H. Fritz, P. Durnez, H.-J. Busch, B. Inderbitzen, D. Barbut, Intra-Arrest Transnasal Evaporative Cooling. Circulation. 122, 729-736 (2010).
[27]. O. D. Kripfgans, J. B. Fowlkes, D. L. Miller, O. P. Eldevik, P. L. Carson, Acoustic droplet vaporization for therapeutic and diagnostic applications. Ultrasound in Medicine and Biology. 26, 1177-1189 (2000).
[28]. P. A. Mountford, M. A. Borden, On the thermodynamics and kinetics of superheated fluorocarbon phase-change agents. Advances in Colloid and Interface Science. 237, 15-27 (2016).
[29]. I. Decosterd, C. J. Woolf, Spared nerve injury: an animal model of persistent peripheral neuropathic pain. Pain. 87, 149-158 (2000).
[30]. X. Gu, Y. Zheng, Y. Cheng, S. Zhong, T. Xi, In vitro corrosion and biocompatibility of binary magnesium alloys. Biomaterials. 30, 484-498 (2009).
[31]. S. W. Hwang, H. Tao, D. H. Kim, H. Cheng, J. K. Song, E. Rill, M. A. Brenckle, B. Panilaitis, S. M. Won, Y. S. Kim, J. A. Rogers, A Physically Transient Form of Silicon Electronics. Science. 337, 1640-1644 (2012).
[32]. S.-K. Kang, R. K. J. Murphy, S.-W. Hwang, S. M. Lee, D. V. Harburg, N. A. Krueger, J. Shin, P. Gamble, H. Cheng, S. Yu, Z. Liu, J. G. McCall, M. Stephen, H. Ying, J. Kim, G. Park, R. C. Webb, C. H. Lee, S. Chung, D. S. Wie, A. D. Gujar, B. Vemulapalli, A. H. Kim, K.-M. Lee, J. Cheng, Y. Huang, S. H. Lee, P. V. Braun, W. Z. Ray, J. A. Rogers, Bioresorbable silicon electronic sensors for the brain. Nature. 530, 71-76 (2016).
[33]. M. N. Nosar, M. Salehi, S. Ghorbani, S. P. Beiranvand, A. Goodarzi, M. Azami, Characterization of wet-electrospun cellulose acetate based 3-dimensional scaffolds for skin tissue engineering applications: influence of cellulose acetate concentration. Cellulose. 23, 3239-3248 (2016).
[34]. I. Rundquist, Q. R. Smith, M. E. Michel, P. Ask, P. A. Oberg, S. I. Rapoport, Sciatic nerve blood flow measured by laser Doppler flowmetry and [14C] iodoantipyrine. American Journal of Physiology-Heart and Circulatory Physiology. 248, H311-H317 (1985).
[35]. G. A. Crowder, Z. L. Taylor, T. M. Reed, J. A. Young, Vapor pressures and triple point temperatures for several pure fluorocarbons. J. Chem. Eng. Data. 12, 481-485 (1967).
[36]. S. G. Lomber, B. R. Payne, J. A. Horel, The cryoloop: an adaptable reversible cooling deactivation method for behavioral or electrophysiological assessment of neural function. Journal of Neuroscience Methods. 86, 179-194 (1999).
[37]. A. Smolyanskaya, R. M. Haefner, S. G. Lomber, R. T. Born, A Modality-Specific Feedforward Component of Choice-Related Activity in MT. Neuron. 87, 208-219 (2015).
[38]. T. Inoue, M. Fujii, H. Kida, T. Yamakawa, Y. Maruta, T. Tokiwa, Y. He, S. Nomura, Y. Owada, T. Yamakawa, M. Suzuki, Epidural focal brain cooling abolishes neocortical seizures in cats and non-human primates. Neuroscience Research. 122, 35-44 (2017).
[39]. D. F. Cooke, A. B. Goldring, I. Yamayoshi, P. Tsourkas, G. H. Recanzone, A. Tiriac, T. Pan, S. I. Simon, L. Krubitzer, Fabrication of an inexpensive, implantable cooling device for reversible brain deactivation in animals ranging from rodents to primates. Journal of Neurophysiology. 107, 3543-3558 (2012).
[40]. D. F. Cooke, I. Stepniewska, D. J. Miller, J. H. Kaas, L. Krubitzer, Reversible Deactivation of Motor Cortex Reveals Functional Connectivity with Posterior Parietal Cortex in the Prosimian Galago (Otolemur garnettii). J. Neurosci. 35, 14406-14422 (2015).
[41]. P. A. Jackson, M. J. Pagliassotti, M. Shiota, D. W. Neal, S. Cardin, A. D. Cherrington, Effects of vagal blockade on the counterregulatory response to insulin-induced hypoglycemia in the dog. American Journal of Physiology-Endocrinology and Metabolism. 273, E1178-E1188 (1997).
[42]. M. A. Long, M. S. Fee, Using temperature to analyse temporal dynamics in the songbird motor pathway. Nature. 456, 189-194 (2008).
[43]. M. A. Long, K. A. Katlowitz, M. A. Svirsky, R. C. Clary, T. M. Byun, N. Majaj, H. Oya, M. A. Howard, J. D. W. Greenlee, Functional Segregation of Cortical Regions Underlying Speech Timing and Articulation. Neuron. 89, 1187-1193 (2016).
[44]. D. Vallentin, G. Kosche, D. Lipkind, M. A. Long, Inhibition protects acquired song segments during vocal learning in zebra finches. Science. 351, 267-271 (2016).
[45]. P. A. Tindall, M. D. Burns, A cooling probe for reversible nerve block. Comparative Biochemistry and Physiology Part A: Physiology. 72, 659-662 (1982).
[46]. I. Mudawar, Assessment of high-heat-flux thermal management schemes. IEEE Transactions on Components and Packaging Technologies. 24, 122-141 (2001).
[47]. A. Bar-Cohen, G. Sherwood, M. Hodes, G. Solbreken, Gas-assisted evaporative cooling of high density electronic modules. IEEE Transactions on Components, Packaging, and Manufacturing Technology: Part A. 18, 502-509 (1995).
[48]. R. M. Guijt, A. Dodge, G. W. K. van Dedem, N. F. de Rooij, E. Verpoorte, Chemical and physical processes for integrated temperature control in microfluidic devices. Lab on a Chip. 3, 1-4 (2003).
[49]. G. Maltezos, A. Rajagopal, A. Scherer, Evaporative cooling in microfluidic channels. Appl. Phys. Lett. 89, 074107 (2006).
[50]. K. W. Ranatunga, B. Sharpe, B. Turnbull, Contractions of a human skeletal muscle at different temperatures. The Journal of Physiology. 390, 383-395 (1987).
[51]. C. McC. Brooks, K. Koizumi, J. L. Malcolm, Effects of changes in temperature on reactions of spinal cord. Journal of Neurophysiology. 18, 205-216 (1955).
[52]. K. H. Chang, C. Pan, Two-phase flow instability for boiling in a microchannel heat sink. International Journal of Heat and Mass Transfer. 50, 2078-2088 (2007).

1. (canceled)

2. (canceled)

3. A device for reversibly blocking activities of a target region of a subject, comprising:
a microfluidic system configured to route a fluid around the target region to change a local temperature of the target region so as to reversibly block activities of the target region,
wherein the microfluidic system is operably in communication with the target region; and
wherein the microfluidic system utilizes a liquid to gas phase transition as a cooling mechanism to change the local temperature of the target region.

4. The device of claim 3, wherein the microfluidic system comprises at least one fluidic chamber formed in a microfluidic layer.

5. The device of claim 4, wherein the at least one fluidic chamber has a length, a footprint and a volume, wherein the length defines a coverage angle of the microfluidic system when wrapping around the target region.

6. The device of claim 4, wherein a cooled area in the target region is confined predominately to a surface directly associated with the at least one fluidic chamber.

7. The device of claim 4, wherein the microfluidic system further comprises transcutaneous colinear interconnects that deliver the fluid to the at least one fluidic chamber in a completely sealed system that provides fluidic access at the ends.

8. The device of claim 7, wherein the microfluidic system further comprises at least first and second input channels and at least one output channel fluidically connected to the at least one fluidic chamber.

9. The device of claim 8, wherein the at least one output channel is colinear to the at least first and second input channels.

10. The device of claim 8, wherein the first and second input channels have widths in ranges of about 50-150 μm and about 200-600 μm, respectively, and the output channel has a width in a range of about 200-600 μm.

11. The device of claim 8, wherein the fluid comprises a coolant and a dry gas being operably transported into the at least one fluidic chamber via the first and second input channels, respectively.

12. The device of claim 11, wherein the microfluidic system is configured such that a simultaneous initiation of the coolant and the dry gas flows into the at least one fluidic chamber prompts evaporation of the coolant at a microfluidic junction between the first and second input channels of the coolant and the dry gas and along the at least one fluidic chamber.

13. The device of claim 12, wherein the at least one fluidic chamber comprises at least one serpentine microfluidic channel formed with a plurality of U-shaped turns over a region in the microfluidic layer.

14. The device of claim 13, wherein the at least one serpentine microfluidic channel operably routes a volume of the coolant to the target region where a flow of the dry gas triggers local and fully contained evaporation of the coolant.

15. The device of claim 14, wherein mass flow rates of the coolant and the dry gas, and the length, the footprint and the volume of the at least one fluidic chamber determine magnitude and localization of the cooling effect.

16. The device of claim 11, wherein the coolant is fluorocarbons, and wherein the dry gas comprises any dry gas including N₂, CO₂, argon, or mixtures thereof.

17. The device of claim 16, wherein the coolant is a bioinert coolant including perfluoropentane (PFP).

18. The device of claim 16, wherein the coolant is a non-bioinert coolant including diethyl ether.

19. The device of claim 8, wherein the microfluidic system further comprises at least one first pump in fluidic communication with the at least first and second input channels for delivering the fluid to the at least one fluidic chamber to change the local temperature of the target region.

20. The device of claim 8, wherein the microfluidic system further comprises at least one second pump in fluidic communication with the at least one output channel for withdrawing the fluid from the at least one fluidic chamber.

21. The device of claim 3, being configured such that a phase change prompts a temperature of the device in a planar, uncurled configuration to drop to about −20° C. within about 2 min or less after initializing flow in ambient, room temperature conditions.

22-51. (canceled)

52. A method for reversibly blocking activities of a target region of a subject, comprising:
routing a fluid around the target region to change a local temperature of the target region so as to reversibly block activities of the target region,
wherein said the fluid around the target region is performed by a microfluidic system that utilizes a liquid to gas phase transition as a cooling mechanism to change the local temperature of the target region.

53. The method of claim 52, wherein the microfluidic system comprises at least one fluidic chamber formed in a microfluidic layer.

54. The method of claim 53, wherein the microfluidic system further comprises at least first and second input channels and at least one output channel fluidically connected to the at least one fluidic chamber.

55. The method of claim 54, wherein the fluid comprises a coolant and a dry gas being operably transported into the at least one fluidic chamber via the first and second input channels, respectively.

56. The method of claim 55, wherein the microfluidic system is configured such that a simultaneous initiation of the coolant and the dry gas flows into the at least one fluidic chamber prompts evaporation of the coolant at a microfluidic junction between the first and second input channels of the coolant and the dry gas and along the at least one fluidic chamber.

57. The method of claim 56, wherein the at least one fluidic chamber comprises at least one serpentine microfluidic channel formed with a plurality of U-shaped turns over a region in the microfluidic layer.

58. The method of claim 57, wherein the at least one serpentine microfluidic channel operably routes a volume of the coolant to the target region where a flow of the dry gas triggers local and fully contained evaporation of the coolant.

59. (canceled)

60. The method of claim 57, wherein said measuring the temperature of the target region is performed by a flexible temperature sensor.

61. The method of claim 57, wherein the flexible temperature sensor is bonded to the microfluidic system.

62. (canceled)

63. A method for fabricating a device for reversibly blocking activities of a target region of a subject, comprising:
fabricating a microfluidic system for routing a fluid around the target region to change a local temperature of the target region; and
fabricating an electronic system coupled with the microfluidic system for providing a real-time feedback,
wherein said fabricating the microfluidic system comprises:
providing a patterned substrate;
coating a sacrificial layer on the patterned substrate;
casting a prepolymer on the coated sacrificial layer and subsequently curing the casted prepolymer to form a polymer layer; and
removing the sacrificial layer from the polymer layer to define at least one fluidic chamber with at least one at least one serpentine microfluidic channel.

64. The method of claim 63, wherein the sacrificial layer is formed of poly(acrylic acid) (PAA).

65. The method of claim 63, wherein the prepolymer is a poly(octanediol citrate) (POC) prepolymer.

66. The method of claim 62, wherein said fabricating the electronic system comprises:
sequentially depositing SiO₂, Mg and SiO₂ onto cellulose acetate to form a temperature sensor layer; and
encapsulating the temperature sensor layer with top and bottom layers of POC.

67. (canceled)
